(19) United States
(12) Patent Application Publication
(10) Pub. No.: US 2019/0077878 A1
(43) Pub. Date: Mar. 14, 2019

(54) METHODS FOR TREATING CANCER BY TARGETING VCAM1 AND MAEA

(71) Applicant: ALBERT EINSTEIN COLLEGE OF MEDICINE, INC., BRONX, NY (US)

(72) Inventors: PAUL S. FRENETTE, NEW YORK, NY (US); SANDRA PINHO, NEW YORK, NY (US); QIAOZHI WEI, BRONX, NY (US)

(73) Assignee: ALBERT EINSTEIN COLLEGE OF MEDICINE, INC., BRONX, NY (US)

(21) Appl. No.: 16/199,555

(22) Filed: Nov. 26, 2018

Related U.S. Application Data
(63) Continuation-in-part of application No. PCT/US2017/034365, filed on May 25, 2017.
(60) Provisional application No. 62/342,360, filed on May 27, 2016.

(51) Int. Cl. C07K 16/30; A61P 35/02; C07K 16/28

(52) U.S. Cl. CPC C07K 16/3061; A61P 35/02; A61K 45/06; C07K 16/2878; C07K 16/2821

(57) ABSTRACT

Methods are disclosed for treating cancers using antibodies and antibody fragments that inhibit the activity of Vascular cell adhesion molecule 1 (Vcam1) and/or Macrophage erythroblast attacher (Maea).

### CROSS-REFERENCE TO RELATED APPLICATIONS

This application is a continuation-in-part of and claims priority of PCT International Patent Application No. PCT/US2017/034365, filed May 25, 2017, which designates the United States of America and which claims the benefit of U.S. Provisional Patent Application No. 62/342,360, filed on May 27, 2016, the contents of which are incorporated herein by reference.

### STATEMENT OF GOVERNMENT SUPPORT

This invention was made with government support under grant numbers HL116340, HL069438 and DK056638 awarded by the National Institutes of Health. The government has certain rights in the invention.

### BACKGROUND OF THE INVENTION

Throughout this application various publications are referred to in superscripts. Full citations for these references may be found at the end of the specification before the claims. The disclosures of these publications are hereby incorporated by reference in their entireties into the subject application to more fully describe the art to which the subject application pertains.

Hematopoietic stem cells (HSCs) possess the ability to maintain the entire population of blood cells throughout life and to replenish the hematopoietic system after transplantation into marrow-ablated recipients. During fetal and adult life, HSCs are able to migrate to ectopic niches via the blood stream. Once in the blood, HSCs home to perivascular stromal and endothelial cells expressing adhesion molecules, then navigate the vascular networks of the marrow, spleen, and liver before returning to potential bone marrow niches.

Under homeostasis, HSCs reside in the specialized bone marrow (BM) niche composed of various cellular and molecular constituents. Whereas mesenchymal stem and progenitor cells provide most niche factor activity contributing to HSC maintenance, differentiated hematopoietic cells such as macrophages can regulate indirectly HSC retention in BM via the niche. In addition, macrophages tightly interact with red blood cell precursors to form a structure known as the erythroblastic island (EI) in which interactions via Vascular cell adhesion molecule 1 (Vcam1) and/or Macrophage-Erythroblast Attacher (Maea, also called EMP) are thought to play important roles in the terminal maturation of erythroblasts. The attachment of the developing erythroblasts (EBs) to the central macrophages within the islands is critical for survival, proliferation and proper differentiation of developing erythrocytes both in vitro and in vivo.

Vcam1 is an adhesion molecule expressed by bone marrow stromal and endothelial cells and certain classes of hematopoietic cells. Vcam1's major ligand is the integrin alpha 4 beta1 (also know as VLA-4). The interaction between Vcam1 and VLA-4 mediates cell-cell interaction in multiple cell types, and both Vcam1 and VLA-4 have been implicated in HSC homing and retention into the bone marrow and mobilization into the peripheral blood.

Maea is an adhesion molecule originally identified on macrophages and erythroblasts and suggested to play a role in the formation of EIs. However, its function in adult hematopoietic system is unknown due to the perinatal death of Maea deficient mice. Other candidate molecules, e.g. Vcam1, have also been suggested to participate in EI formation, but cell type-specific requirement of these molecules for EI formation and function in vivo has not been examined.

The present invention provides anti-Vcam1 and anti-Maea therapies for treatment of hematological malignancies and other cancers.

### SUMMARY OF THE INVENTION

The present invention provides methods of treating a cancer (e.g., a hematologic malignancy) in a subject comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) to treat a cancer in a subject, wherein the antibody or antibody fragment is specific for Vcam1 or Maea.

Also provided are methods of inhibiting engraftment of leukemia cells (e.g., acute myeloid leukemia (AML), chronic myeloid leukemia (CML), acute lymphoblastic leukemia (ALL), or chronic lymphocytic leukemia (CLL) cells) in a subject, the methods comprising administering to the subject an antibody or antibody fragment in an amount effective to inhibit the activity of Vcam1 and/or an antibody or antibody fragment in an amount effective to inhibit the activity of Maea to inhibit leukemia (e.g., AML, CML, ALL or CLL) cell engraftment in a subject, wherein the antibody or antibody fragment is specific for Vcam1 or Maea.

Still further provided are methods of enhancing the efficacy of cytarabine for treating a cancer in a subject, comprising administering to the subject an antibody or antibody fragment in an amount effective to inhibit the activity of Vcam1 and/or an antibody or antibody fragment in an amount effective to inhibit the activity of Maea in combination with cytarabine to enhance the efficacy of cytarabine for treating a cancer in a subjectb wherein the antibody or antibody fragment is specific for Vcam1 or Maea.

### BRIEF DESCRIPTION OF THE DRAWINGS

FIG. 1A-1C. Vcam1 is expressed at higher levels on acute myelogenous leukaemia (AML) stem cells than on healthy hematopoietic stem cells. (A) Percentage of Vcam1⁺ cells within hematopoietic stem cells (HSC) and multipotent progenitors (MPP) from the bone marrow (BM), spleen and blood (n=6-18). (B) Outline of experiment strategy to generate mouse leukemic MLL-AF9 cells. (C) Median fluorescence intensity (MFI) of Vcam1 on bulk control healthy and leukemic MLL-AF9 BM cells (left panel), and healthy HSCs and leukaemia stem cells (LSCs, right panel). LSCs were phenotypically defined as Lineage-IL7Rα-Sca1-MLL-AF9 GFP+ c-Kit^{high} CD34^{low} FcγRII/III^{high} cells. (n=6-9); MPP (LSK CD150⁻ CD48⁻); HSC (LSK CD150⁺ CD48⁻).

FIG. 2A-2G. Vcam1 endogenous deletion does not cause significant hematopoietic defects. (A) FACS analysis of the BM of Csflr-iCre; loxp-TdTomato transgenic mice showing the recombination efficiency of Csflr-iCre in phagocytes, HSC and MPP (n=3-6). (B) Vcam1 is efficiently depleted in Vcam^{Δ/Δ} BM HSCs, as seen by FACS (n=4-13) and mRNA (n=4-6) analyses. (C) Absolute number of BMNCs, HSCs and MPPs per femur in control and Vcam1^{Δ/Δ} mice (n=5-6). (D) Colony output on day 7 of BM colony-forming unit in culture from control and Vcam1^{Δ/Δ} mice. GEMM: granulocyte, macrophage, erythroid and megakaryocyte; GM: granulocyte and macrophage; M: macrophage; G: granulocyte; BFU-E: erythroid. (n=3). (E) Concentration of white blood cells (WBC), erythrocytes (RBC) and platelets (PLTs) in the blood of Vcam1^{Δ/Δ} mice as compared to littermate controls (n=12). (F) Concentration of HSCs and MPPs in the blood of Vcam1^{Δ/Δ} mice as compared to littermate Vcam1 floxed controls (n=3). (G) Spleen cellularity (left graph) and absolute number of HSC and MPP (right graph) per spleen in control and Vcam1^{Δ/Δ} mice (n=5). Error bars, mean±s.e.m. *P<0.05, **P<0.01, ****P<0.0001; Unpaired student's t test.

FIG. 3A-3E. Vcam1-deficient HSCs exhibit normal viability, cell cycle and proliferation. (A) Percentage of viable (Annexin V⁻ DAPI⁻) HSC and MPP in the BM of control and Vcam1^{Δ/Δ} mice. (n=3). (B) Cell cycle analysis, using anti-Ki67 and Hoechst 33342 staining, of HSCs from control and Vcam1^{Δ/Δ} mice (n=3-4). (c) Percentage of proliferating HSC in the BM of control and Vcam1^{Δ/Δ} mice, as determined by BrdU incorporation (n=4). (D) Quantitative PCR (qPCR) analysis of cell cycle regulator genes within sorted HSCs from control and Vcam1^{Δ/Δ} mice. (E) Number of BMNCs, MPP and HSC per femur in control and Vcam1^{Δ/Δ} mice after 5-FU injection (n=3-5). Error bars, mean s.e.m. Non-significant (ns); *P<0.05. Unpaired student's t test (A-E).

FIG. 4A-4E. Blocking anti-Vcam1 antibody treatment decreases the number of leukaemia stem cells and synergizes with cytarabine in vivo. (A) Outline of experiment strategy. Moribund sick secondary recipient leukemic mice were daily injected with IgG control (100 μg), anti-Vcam1 antibody (100 μg), cytarabine (100 mg/kg) or a combination of anti-Vcam1/cytarabine during 5 days. Mice were analysed by FACS 1 day after the last injection. (B) BM cellularity, absolute number and percentage of bulk MLL-AF9-GFP⁺ cells and LSCs in the BM of control and treatment groups. (n=5-6). (C) Percentage of MLL-AF9-GFP⁺ cells in the blood of recipient hosts comparing pre- and post-treatment. (n=5) (D) Survival curves of leukemic treatment groups. Arrow points to the beginning of treatment. IgG and anti-Vcam1 were administered during 10 consecutive days every and cytarabine groups during 5 consecutive days. All treatments were repeated after 4 weeks. (E) BM cellularity (left graph) and absolute number of HSC, MPP and LSK per femur (middle graph) and per ml of blood (left graph) in healthy C57BL/6 mice treated for 5 days with daily injections of either anti-Vcam1 or IgG control antibody (100 μg) (n=5). Error bars, mean±s.e.m. *P<0.05, **P<0.01, ***P<0.001, ****P<0.0001; Unpaired student's t test (E) and paired student's t test (C). One-way ANOVA analyses followed by Tukey's multiple comparison tests were in (B). Log-rank analysis was used for the Kaplan-Meier survival curves in (D).

FIG. 5A-5D. Treatment of healthy wild-type mice with a blocking anti-Vcam1 monoclonal antibody. (A) Outline of experiment strategy. (B) BM cells from treated groups in (B) were incubated with an anti-rat antibody and after washing stained for phenotypic HSCs and probed for Vcam1 expression. (C) Body, liver and spleen weight of IgG and anti-Vcam1-treated mice from (A). (D) Peripheral blood was drawn post-treatment and hematology lab analysis was performed. White blood cell (WBC), red blood cell (RBC), hemoglobin (HGB), hematocrit (HCT), platelets (PLT), neutrophils (Neut.), lymphocytes (Lymph.), reticulocytes (Retic.). P-values of IgG compared to anti-Vcam1 treated mice are shown for each parameter (n=5). Error bars, mean ±s.e.m. Non-significant (ns). Unpaired student's t test.

FIG. 6A-6D. High VCAM1 expression is associated with poor prognosis in human AML patients. (A, B) Kaplan-Meier (A) overall and (B) disease free survival of AML patients (TCGA, Ley et al., 2013) with high and low VCAM1 expression (mRNA expression z-Score threshold±2). (C-D) Survival curves of NSG mice transplanted with primary human AML samples and treated with (C) control IgG or anti-VCAM1 antibody or (D) with cytarabine or the combination cytarabine/anti-VCAM1. Log-rank analysis was used for the Kaplan-Meier survival curves to calculate P value.

FIG. 7A-7B. VCAM1 expression in human cancer cell lines. (A) Pie chart shows VCAM1 expression status across 675 human cancer cell lines. (B) Mean distribution of VCAM1 expression (reads per kilobase of transcript per million mapped reads—RPKM) per human cancer cell line, grouped by metastatic tissue of origin⁴⁵.

FIG. 8. VCAM1 genetic alterations in primary human cancer tissues. Cross-cancer alteration summary for VCAM1 from 126 human cancer genomics studies generated by cBioPortal for Cancer Genomics from MSKCC.

FIG. 9A-9G. Deletion of Maea impairs bone marrow macrophage development and the erythroblastic island. (A) Representative histogram showing Maea expression on BM leukocytes from Maea^{fl/fl} control and Maea^{Csflr-Cre} mice. (B) Deletion efficiency by Csflr-Cre of Maea on bone marrow macrophages as determined by FACS (n=8). (C) Quantification of total BM cellularity in Maea^{fl/fl} and Maea^{Csflr-Cre} mice (n=8). (D, E) Representative FACS plots and quantification showing significant reduction of macrophage (D) and erythroblast (E) numbers in the bone marrow of Maea^{Csflr-Cre} mice compared to littermate control (n=8). (F) Quantification of erythroblasts at various stages of maturation (subpopulation I-V represents: I: proerythroblasts, II: basophilic erythroblasts, III: polychromatic erythroblasts, IV: orthochromatic erythroblasts and reticulocytes, V: mature RBCs) (n=8). (G) RBC counts of Maea^{fl/fl} and Maea^{Csflr-Cre} mice (n=10). Data are shown as mean±s.e.m. *p<0.05, **p<0.01, ***p<0.001, ****p<0.0001 by unpaired Student's t test.

FIG. 10A-10H. Deletion of Maea impairs bone marrow macrophage development and erythroblastic niche. (A) Schematic representation of the Maea^{Targeted} allele, Maea^{floxed} allele and Maea^{delta} allele generated using EuMMCR targeting vector PG00141_Z_1_G10. Exons are depicted by boxes with coding regions indicated by shading. FRT sites are marked as white triangles and LoxP sites as black triangles. The IRES-LacZ reporter (LacZ) and the neomycin resistance cassette (Neo) were deleted by crossing with a Flpe-expressing deleter strain. Upon tissue-specific or temporal Cre recombinase induction, the Maea exon 5 will be deleted which will result in a null Maea^{delta} allele caused by non-sense mediated decay. (B) PCR analysis identifying the wild-type (WT), Maea^{Floxed} and Maea^{Targeted} allele. (C) Representative FACS plots and quantification showing impaired in vivo formation of BM erythroblastic islands (F4/80+ Ter119+ live multiplets) in Maea^{Csflr-Cre} mice (n=5). (D) WrightGiemsa stained smears from control and Maea^{Csflr-Cre} peripheral blood. Scale bar=50 μm. (E) Quantification of spleen erythroblasts in Maea^{Csflr-Cre} mice (n=8). (F) Burst-forming unit-erythroid (BFU-E) in Maea^{Csflr-Cre} spleen (n=5). (G) Representative histograms and quantification shown prolonged half-life of in vivo biotinylated RBCs in Maea^{Csflr-Cre} mice (n=5). (H) RBC counts and HCT in splenectomized control and Maea^{Csflr-Cre} mice (n=5). Data are shown as mean±s.e.m. *p<0.05, **p<0.01, ***p<0.001, ****p<0.0001 by unpaired Student's t test.

FIG. 11A-11J. Maea is required for lymphoid differentiation. (A) WBC counts in Maea^{fl/fl} and Maea^{Csflr-Cre} mice (n=10). (B) Frequency of B, T and myeloid cells in total WBCs of Maea^{fl/fl} and Maea^{Csflr-Cre} (n=5). (C, D) Number of B cells (C) and B progenitor subsets (D) in the BM of control and Maea^{Csflr-Cre} mice (n=5). (E) Representative histograms showing Maea expression on Maea^{fl/fl} HSPCs and deletion on Maea^{Csflr-Cre} HSPCs. (F) Quantification of deletion efficiency of Maea expression on HSPCs by Csflr-Cre (n=4). (G) LSK and HSC numbers in BM of control and Maea^{Csflr-Cre} mice (n=8). (H) Quantification of progenitors in BM of control and Maea^{sflr-Cre} mice; number of common myeloid progenitors (CMP), granulocyte-macrophage progenitors (GMP), megakaryocyte-erythroid progenitors (MEP) and megakaryocyte progenitors (MkP). (I, J) Representative FACS plots and quantification of lymphoid-primed multipotent progenitors (LMPP) (I) and common lymphoid progenitors (CLP) (J) subsets in BM of control and Maea^{Csflr-Cre} mice (n=6). Data are shown as mean±s.e.m. *p<0.05, **p<0.01, ***p<0.001, ****p<0.0001 by unpaired Student's t test.

FIG. 12A-12H. Maea is required for HSC engraftment. (A) Reconstitution capability of Maea^{fl/fl} and Maea^{Csflr-Cre} HSCs as determined by competitive BM transplantation (n=5). 1×10⁶ of donor (CD45.2) BM cells were competitively transplanted with equal number of competitor (CD45.1) BM cells into lethally irradiated recipient mice (CD45.1). Percentage of donor derived B220+B, CD3+ T and CD11b+Gr1+ myeloid cells were quantified at indicated time points. (B) Experimental design of the reciprocal BMT performed. (C) 16 weeks after the transplant, percentage of donor derived cells were quantified in the BM, peripheral blood and spleen of the control and Maea^{Csflr-Cre} recipients. (D, E) WBC counts (D) and BM cellularity (E) in control and Maea^{Csflr-Cre} recipient mice 16 weeks after the transplant (n=5). (F) Frequency of B, T and myeloid cells in total WBCs of control and Maea^{Csfr-Cre} reciprocal recipients (n=5). (G, H) Quantification of BM macrophages (g) and erythroblasts (h) control and Maea^{Csflr-Cre} reciprocal recipients (n=5). Data are shown as mean±s.e.m.

FIG. 13A-13 F. Maea is required for HSC engraftment but dispensable for their maintenance. (A) Cell cycle analysis of BM HSCs by Ki-67 and H33342 dye staining (n=3). (B) Cleaved caspase3 expression in BM LSKs from the control and Maea^{Csfr-Cre} mice (n=3). (C) Assessment of peripheral blood recovery of the control and Maea^{Csfr-Cre} mice after 250 mg/kg of 5-FU challenge (n=6). (D) BM total cellularity, LSK and HSC numbers of the control and Maea^{Csfr-Cre} mice 4 weeks after 5-FU injection. (E) Quantification of homed BMNCs and LK cells from control and Maea^{Csflr-Cre} mice in lethally irradiated WT CD45.1 recipients (n=5). (F) Comparable differentiation potential of control and Maea^{Csflr-Cre} LSK cells measured by colony-forming assays (n=4).

FIG. 14A-14D. MAEA over-expression is associated with poor prognosis of human cancers. (A) Cross-cancer alteration summary for MAEA from 126 human cancer genomics studies generated by cBioPortal for Cancer Genomics from MSKCC. (B) Kaplan-Meier overall and disease free survival of AML patients (TCGA, NEJM 2013) with high and low MAEA expression (mRNA expression z-Score threshold±2). (C, D) Kaplan-Meier overall survival of ovarian cancer and lung adenocarcinoma patients (TCGA) with high and low MAEA expression (mRNA expression z-Score threshold±2). The significance is based on log rank test estimate of p values.

FIG. 15A-15J. MAEA is required for mouse AML engraftment and progression. (A) schematic development of MLL-AF9 acute myeloid leukemia (AML) model. (B) expression level of MAEA, measured by mean fluorescent intensity (MFI), in total bone marrow cells (BM), LSK, lineage-ckit+ (LK) and granulocyte-macrophage progenitors (GMP) of healthy and AML mice. In leukemic mice, both GFP+ AML cells (AML) and their residual GFP− healthy counterparts (non-AML) were assessed. (C) quantification of GFP+ AML cells in primary sub-lethally irradiated recipients that received Ctrl and Maea^{Csflr-Cre-} pre-leukemic cells. (D) Assessment of the peripheral blood of mice transplanted with control and Maea^{Csflr-Cre} pre-leukemic cells at 1012 weeks after transplant. PLT, platelet. (E) Survival curve of mice transplanted with control and Maea^{Csflr-Cre} pre-leukemic cells (n=5). p value is determined by Log-rank test. (F) Representative FACS analysis of BM cells from control and Maea^{Csflr-Cre} pre-leukemic mice at 10˜12 weeks after transplant. (G, H) Quantification of total leukaemia load (GFP+) (G) and leukemic GMP (L-GMP) (H) in recipients of control and Maea^{Csfr-Cre} pre-leukemic cells at 10˜12 weeks after transplant (n=5). (I) Progression of circulating control and Maea^{Mxl-cre} AML cells after a single injection of pIpC (arrow). (J) Progression of circulating wild type AML cells after injections of 60 μg anti-MAEA polyclonal antibody (arrows). Data are shown as mean±s.e.m. *p<0.05, **p<0.01, ***p<0.001, ****p<0.0001 by unpaired Student's t test.

FIG. 16A-16E. Wild type mice treated with IgG and anti-MAEA antibody. Wild type mice were given three doses of 60 μg IgG and anti-MAEA antibody i.v. every other day and analysed 2 days after the last injection. (A-D) Total body, spleen and liver weight (A), BM and spleen cellularity (B), erythroblasts percentage in the BM (C) and LSK and HSC percentages in the BM and spleen (D) of IgG and anti-MAEA antibody treated mice. (E) Summary of peripheral blood parameters from mice treated with IgG and anti-MAEA antibody. WBC, white blood cells. RBC, red blood cells. HGB, haemoglobin. HCT, haematocrit. MCV, mean corpuscular volume. PLT, platelets. Retic, reticulocyte. Lymph, lymphocyte. Data are shown as mean±s.e.m. *p<0.05, **p<0.01 by unpaired Student's t test.

FIG. 17A-17D. MAEA expression in human cancer cell lines. RNA-seq data of 675 human cancer cell lines across tissue types were previously published⁴⁵and made available on the web at research-pub.gene.com/KlijnEtA12014. (A) Distribution of MAEA mRNA expression (RPKM) across all 675 lines. (B) MAEA expression in cancer cell lines across their tissue origin. (C, D) MAEA expression in lung (C) and ovarian (D) cancer cell lines.

FIG. 18A-18F Maea maintains adult BM erythroblastic island niche. (A) Validation of the specificity of mAb produced by hybridoma clone 92.25 by FACS staining of BM macrophages from wild type control and Maea^{Csflr-Cre} mice (B-E) Quantification of total BM cellularity (B), BM EB numbers (C), EB maturation profile (D), and BM macrophage numbers (E) in isotype or anti-MAEA mAb-treated mice (n=5). (F) Quantification of erythroid cells per erythroblastic island reconstituted in the presence of 10 μg/ml isotype or anti-MAEA mAb. Data are shown as mean±s.e.m. *p<0.05, **p<0.01, ****p<0.0001 by unpaired Student's t test.

### DETAILED DESCRIPTION OF THE INVENTION

The present invention provides a method of treating a cancer in a subject comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) to treat a cancer in a subject, wherein the antibody or antibody fragment is specific for Vcam1 or Maea.

As used herein, the term “treat” a cancer means to eradicate the cancer in a subject, or to reduce the size of a cancer or cancer tumor in the subject, or to stabilize a cancer or cancer tumor in the subject so that it does not increase in size, or to prevent or reduce the spread of the cancer in the subject.

The cancer can be, for example, one or more of bladder, breast, brain, colorectal, kidney, esophagus, gastrointestinal tract, liver, lung, ovarian, pancreas, prostate, skin, stomach, and uterine cancer, melanoma, non-Hodgkin lymphoma, myelodysplatic syndrome (MDS) (a pre-leukemia), and a hematologic malignancy. Hematologic malignancies can derive from myeloid or lymphoid cell lines. Lymphomas, lymphocytic leukemias, and myeloma are from the lymphoid line, while acute and chronic myelogenous leukemia, myelodysplastic syndromes and myeloproliferative diseases are myeloid in origin. The hematologic malignancy can be a myeloproliferative disease. The hematologic malignancy can be, for example, acute myeloid leukemia (AML), chronic myeloid leukemia (CML), acute lymphoblastic leukemia (ALL), or chronic lymphocytic leukemia (CLL).

The treatment can comprise administering to a subject a combination of two or more of:
a) a blocking antibody to Vcam1 or an antibody fragment that blocks the activity of Vcam1, wherein the antibody or antibody fragment is specific for Vcam1;
b) a blocking antibody to Maea or an antibody fragment that blocks the activity of Maea, wherein the antibody or antibody fragment is specific for Maea;
c) one or more chemotherapeutic agents; and
d) one or more immune system enhancing agents;
wherein the combination includes at least a) or b).

The different components of the combination can be administered at the same time, sequentially, or one spaced in time before the other.

The one or more chemotherapeutic agents can be, for example, but not limited to, cytarabine (cytosine arabinoside or ara-C), an anthracycline drug (such as, e.g., daunorubicin (daunomycin), idarubicin, and/or mitoxantrone), cladribine (2-CdA), fludarabine (Fludara®), topotecan, etoposide (VP-16), 6-thioguanine (6-TG), hydroxyurea (Hydrea®), a corticosteroid drug (such as, e.g., prednisone or dexamethasone (Decadron®)), methotrexate (MTX), 6-mercaptopurine (6-MP), azacitidine (Vidaza®), and/or decitabine (Dacogen®).

The one or more immune system enhancing agents can be, for example, but not limited to, an inhibitor of CD47 (also called Cluster of Differentiation 47 and integrin associated protein (TAP)), PD-1 (also called Programmed cell death protein 1) /PD-L1 (also called Programmed death-ligand 1, Cluster of Differentiation 274 (CD274) and B7 homolog 1 (B7-H1)), CTLA-4 (also called cytotoxic T-lymphocyte-associated protein 4 and CD152 (Cluster of Differentiation 152)), CD200 (also called Cluster of Differentiation 200 or OX-2 membrane glycoprotein)/CD200R (CD200 reseptor), LAG-3 (also called Lymphocyte-activation gene 3 protein), TIM-3 (also called T-cell immunoglobulin and mucin-domain containing-3), VISTA (also called V-domain Ig suppressor of T cell activation), or TIGIT (also called T cell immunoreceptor with Ig and ITIM domains). The agent that inhibits the activity of, for example, CD47 can be, for example, a blocking antibody to CD47 or an antibody fragment that blocks the activity of CD47, where the antibody or antibody fragment is specific to CD47. Examples of blocking antibodies to CD47 are described in US2016/0137733, US2016/0137734 and US2017/0081407, hereby incorporated by reference. The agent that inhibits the activity of CD47 can also be a construct having a SIRP alpha domain or variant thereof. Such constructs are described, for example, in US2015/0071905, US2015/0329616, US2016/0177276, US2016/0186150 and US20170107270, hereby incorporated by reference.

Also provided is a method of inhibiting engraftment of leukemia cells in a subject, the method comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) to inhibit leukemia cell engraftment in a subject, wherein the antibody or antibody fragment is specific for Vcam1 or Maea. The leukemia cells can be, for example, acute myeloid leukemia (AML), chronic myeloid leukemia (CML), acute lymphoblastic leukemia (ALL), or chronic lymphocytic leukemia (CLL) cells.

Still further provided is a method of enhancing the efficacy of cytarabine for treating a cancer in a subject, comprising administering to the subject an antibody or antibody fragment in an amount effective to inhibit the activity of Vcam1 and/or an antibody or antibody fragment in an amount effective to inhibit the activity of Maea in combination with cytarabine to enhance the efficacy of cytarabine for treating a cancer in a subject, wherein the antibody or antibody fragment is specific for Vcam1 or Maea. The cancer can be, for example, one or more of AML, CML, ALL, CLL and non-Hodgkin's lymphoma.

Preferably, the antibody or antibody fragment is an antagonistic or blocking antibody or fragment.

The antibody or antibody fragment that specifically inhibits the activity of Vcam1 is preferably a blocking antibody to Vcam1 or an antibody fragment that blocks the activity of Vcam1. The antibody or antibody fragment that specifically inhibits the activity of Maea is preferably a blocking antibody to Maea or an antibody fragment that blocks the activity of Maea.

As used herein, the term “antibody” refers to an intact antibody, i.e. with complete Fc and Fv regions. Antibody “fragment” refers to any portion of an antibody, or portions of an antibody linked together, such as, in non-limiting examples, a Fab, F(ab)₂, a single-chain Fv (scFv), which is less than the whole antibody but which is an antigen-binding portion and which competes with the intact antibody of which it is a fragment for specific binding to the target. As such a fragment can be prepared, for example, by cleaving an intact antibody or by recombinant means.

In preferred embodiments, the antibody is a monoclonal antibody. A monoclonal antibody to Maea is available from R&D Systems (MAB7288), and a recombinant mouse monoclonal antibody to human Maea is available from Creative Biolabs. Vcam1 monoclonal antibodies are available from, e.g., Thermo Fisher Scientific, Abcam, Sigma-Aldrich, and Abnova. Vcam1 monoclonal antibodies are also described in US2010/0172902, incorporated herein by reference.

The antibody can be a human antibody or a humanized antibody or a chimeric antibody. As used herein, a “human antibody” unless otherwise indicated is one whose sequences correspond to (i.e. are identical in sequence to) an antibody that could be produced by a human and/or has been made using any of the techniques used for making human antibodies, but not one which has been made in a human. “Chimeric antibodies” are forms of non-human (e.g., murine) antibodies that contain human sequences in the constant domain regions of the antibody in order to eliminate or reduce immunogenic effects. “Humanized” forms of non-human (e.g., murine) antibodies are chimeric antibodies that also contain human sequences in the variable domain regions of the antibody and thus contain minimal sequence derived from non-human immunoglobulin. In general, a humanized antibody can comprise substantially all of at least one, and typically two, variable domains, in which all or substantially all of the hypervariable loops correspond to those of a non-human immunoglobulin, and all or substantially all of the framework regions are those of a human immunoglobulin sequence. In one embodiment, a humanized antibody is a human immunoglobulin (recipient antibody) in which residues from a hypervariable region (HVR) of the recipient are replaced by residues from a HVR of a non-human species (donor antibody) such as mouse, rat, rabbit, or nonhuman primate having the desired specificity, affinity, and/or capacity. For example, the antibody to Maea could be a human or humanized antibody having the CDRs of MAB7288 (which is a mouse IgG1 Ab).

Also provided is a monoclonal antibody to Maea.

The antibody or antibody fragment can be administered to the subject in a pharmaceutical composition comprising the antibody or fragment and a pharmaceutically acceptable carrier. The term “carrier” is used in accordance with its art-understood meaning, to refer to a material that is included in a pharmaceutical composition but does not abrogate the biological activity of the antibody or antibody fragment included within the composition. Pharmaceutically acceptable carriers include, for example, sterile isotonic saline, phosphate buffered saline solution, water, and emulsions, such as an oil/water or water/oil emulsions.

The antibody or antibody fragment can be administered to subjects using routes of administration known in the art, including, but are not limited to, intravenous, intramuscular and intraperitoneal administration.

Also provided are a blocking antibody to Vcam1, an antibody fragment that blocks the activity of Vcam1, a blocking antibody to Maea, and an antibody fragment that blocks the activity of Maea for use as a medicament in treatment of cancer, in inhibiting engraftment of leukemia cells such as AML, CML, ALL and CLL cells, and in enhancing the efficacy of cytarabine for treatment of cancer, wherein the antibody or antibody fragment is specific for Vcam1 or Maea. The cancer can be, for example, one or more of bladder, breast, brain, colorectal, kidney, esophagus, gastrointestinal tract, liver, lung, ovarian, pancreas, prostate, skin, stomach, and uterine cancer, melanoma, myelodysplatic syndrome (MDS) (a pre-leukemia), non-Hodgkin lymphoma, and a hematologic malignancy. Hematologic malignancies can derive from myeloid or lymphoid cell lines. Lymphomas, lymphocytic leukemias, and myeloma are from the lymphoid line, while acute and chronic myelogenous leukemia, myelodysplastic syndromes and myeloproliferative diseases are myeloid in origin. The hematologic malignancy can be a myeloproliferative disease. The hematologic malignancy can be, for example, AML, CML, ALL or CLL.

The antibody or antibody fragment can be conjugated with a cytotoxic agent.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Example I

### Anti-Vcam1 Therapies

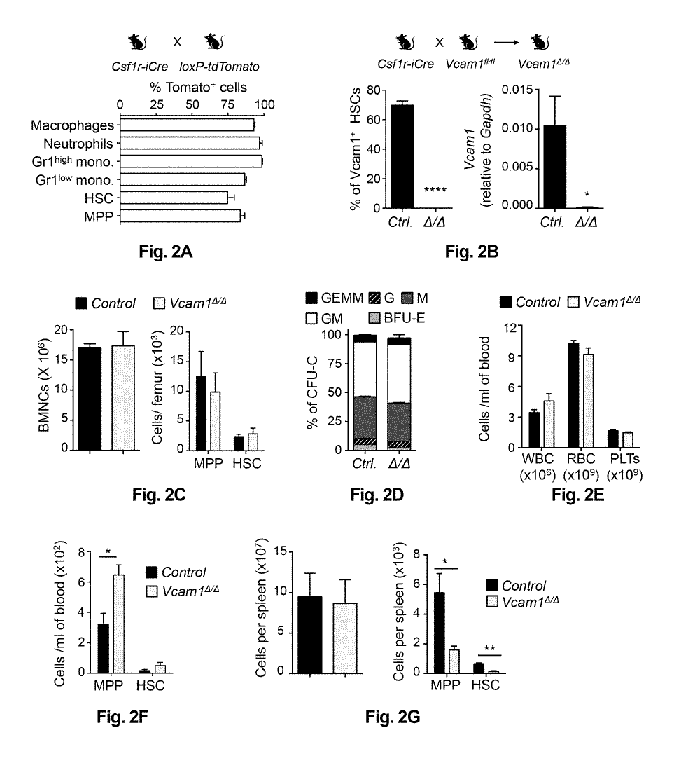

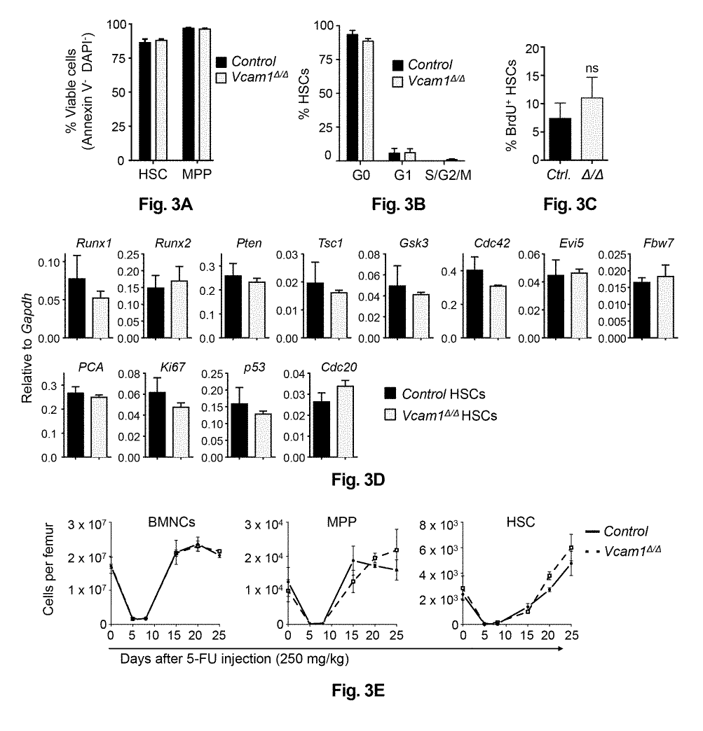

Vcam1 is expressed on hematopoietic stem and progenitor cells (HSPCs, FIG. 1A). Although Vcam1 expression in endothelial cells and its functional implications have been extensively described, the role of Vcam1 on HSCs has not been explored. Recent studies also suggest that Vcam1 expression on endothelial and bone marrow (BM) stromal cells may mediate in part leukemic cell resistance to conventional chemotherapy. Vcam1 is more highly expressed on acute myelogenous leukemia (AML) cells than their healthy counterparts (FIG. 1B-FIG. 1C). Since Csflr-iCre mice exhibit broad Cre expression in all hematopoietic cells, including most HSCs (FIG. 2A) and deletion of Vcam1 gene is embryonically lethal, Vcam1 floxed mice were bred with a Csflr-icre transgenic line (referred to as Vcam1^{Δ/Δ}) to investigate Vcam1's function postnatally. In this model Vcam1 was efficiently depleted in phagocytic cells and also HSCs (FIG. 2B). Vcam1 deletion in Csflr-icre+ cells induced HSPC mobilization into the peripheral blood; however, it did not significantly impair hematopoiesis (FIG. 2C-FIG. 2G). Vcam1 deletion did not increase the number of apoptotic HSCs as determined by Annexin V staining (FIG. 3A) and no significant changes were observed in the proportion of cycling HSCs or genes involved in HSC quiescence/proliferation (FIG. 3B-FIG. 3D). Vcam1 Δ/Δ and control mice challenged with 5-FU did not reveal any deficit in hematopoietic stem and progenitor recovery (FIG. 3E).

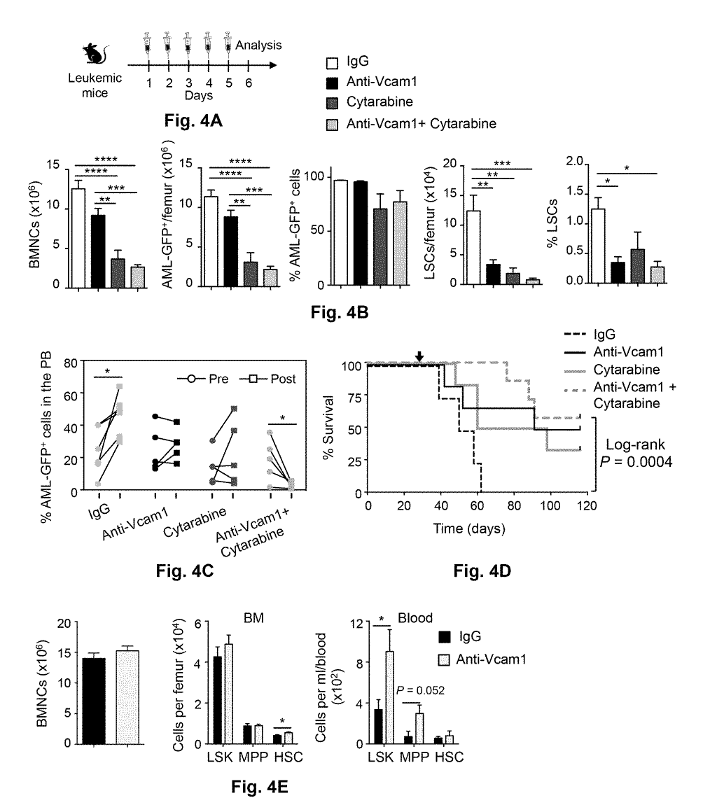

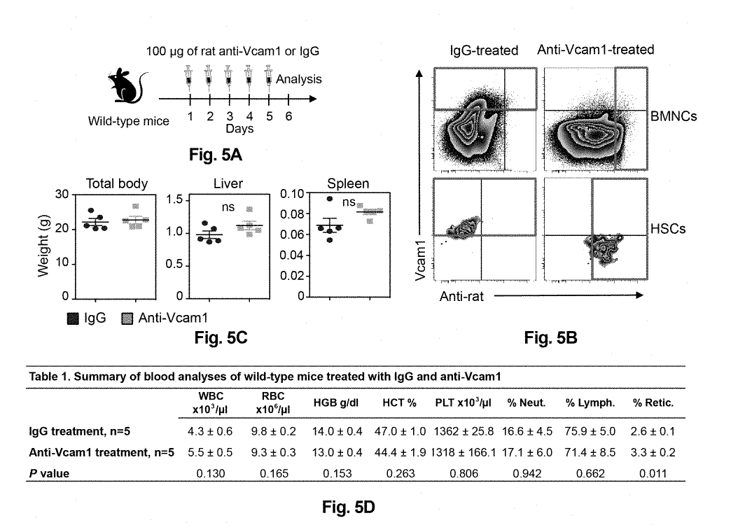

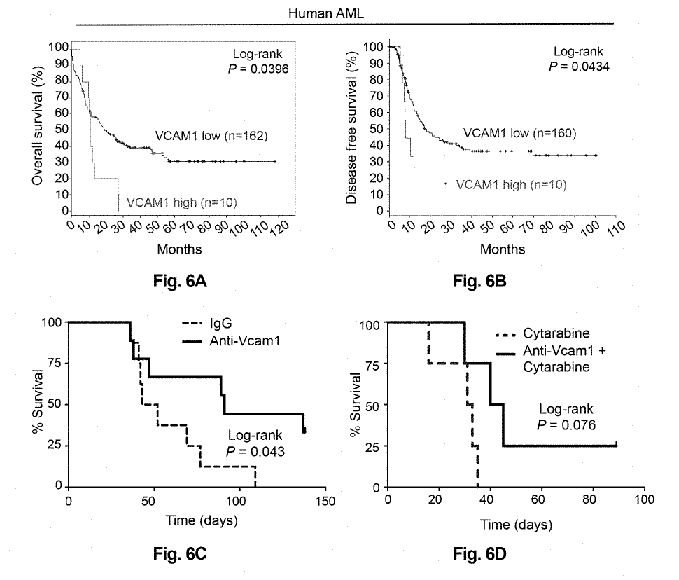

To test whether Vcam1 antibody blockade can improve conventional chemotherapy in animals with established disease, AML was established in immunocompetent C57BL/6 recipients and then therapy of moribund leukemic mice was initiated with a daily injection of IgG control, anti-Vcam1, cytarabine, or a combination of anti-Vcam1/cytarabine. Anti-Vcam1 antibody inhibition synergised with conventional chemotherapy to clear leukemic stem cells (LSCs) while sparing healthy HSCs, significantly prolonging mice survival (FIG. 4). The viability of targeting Vcam1 as a therapeutic strategy was investigated by injecting healthy wild-type mice with anti-Vcam1 antibody. After treatment, mice appeared healthy and body, liver and spleen weighs were unaltered (FIG. 5A-FIG. 5C). Complete blood counts showed no hematopoietic defects but did indicate a small increase in the percentage of reticulocytes (FIG. 5D). These results indicate that targeting Vcam1 function with a blocking monoclonal antibody should be well tolerated and a promising therapeutic strategy. Analysis of The Cancer Genome Atlas (TCGA) databases indicated that high VCAM1 expression was associated with poor prognosis in human AML patients (FIG. 6A-FIG. 6B). Furthermore, anti-VCAM1 treatment was able to significantly extend the survival of immunocompromised mice transplanted with human primary AML samples (FIG. 6C).

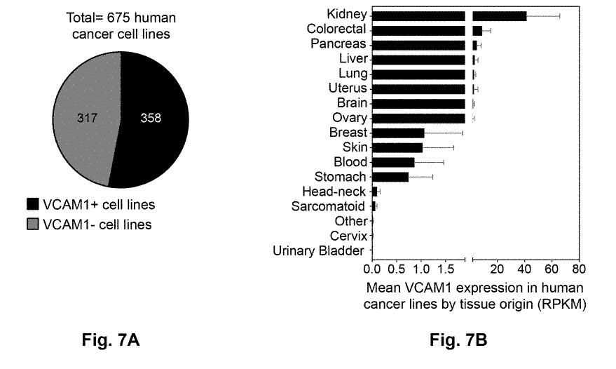

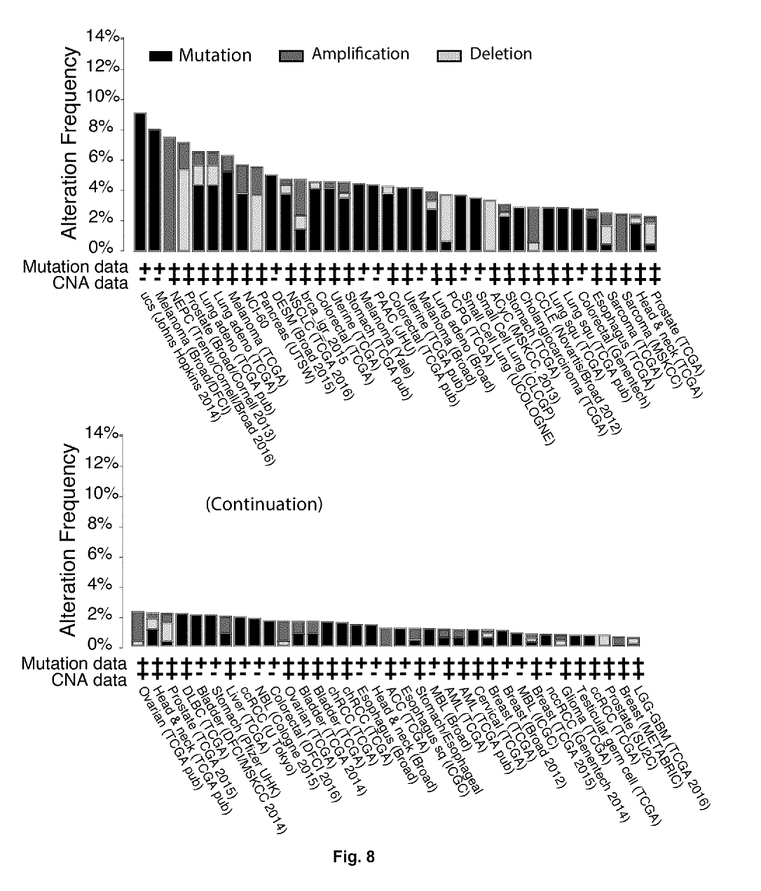

Analysis of a recently published RNA-sequencing dataset of 675 human cancer cell lines indicated that >50% of those lines express VCAM1 (FIG. 7A). In fact, different tissue types of human cancer cell lines express high levels of VCAM1, in particular kidney, colorectal and pancreas (FIG. 7B)⁴⁵, and significant association of VCAM1 gene alterations were found with many human cancer types (FIG. 8).

These studies demonstrate that Vcam1 is unregulated on malignant hematopoietic cells and that inhibition of binding of Vcam1 to its receptors will promote cancer cell clearance. These studies also indicate that this cell clearance mechanism is likely via a “don't-eat-me” signal since incubation of Vcam1 Δ/Δ AML cells with macrophages led to enhanced phagocytosis of leukemic cells. This effect did not result from a reduced expression of CD47, since CD47 expression was not altered in Vcam1 Δ/Δ mice. Monoclonal antibodies either alone or in combination with treatment such as cytarabine are an effective treatment for cancer.

### Example II

### Anti-Maea Therapies

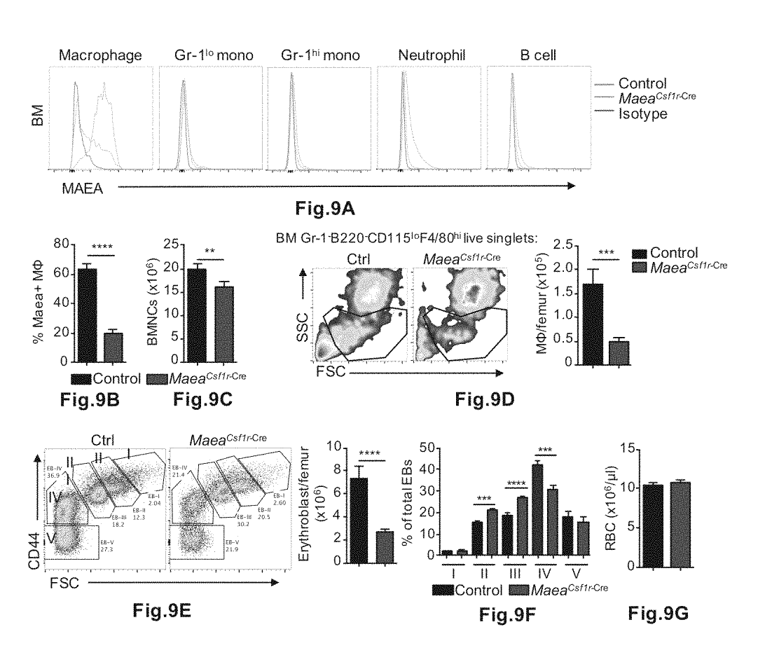

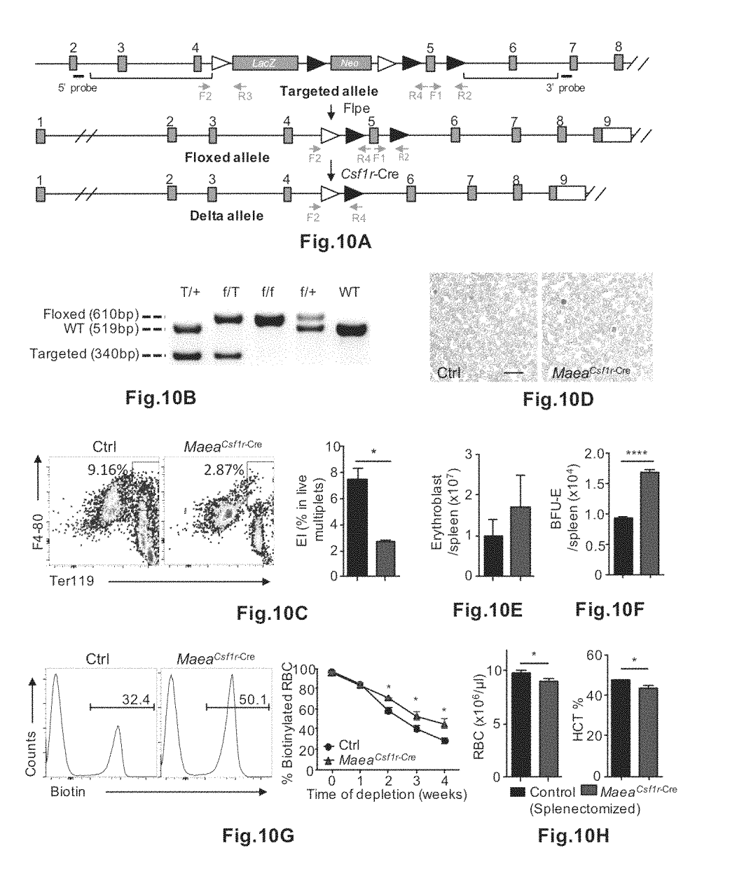

Conditional Maea knockout (Maea^{floxed}) mice were generated and macrophage Maea expression deleted by Csflr-Cre (FIG. 9A, FIG. 9B, FIG. 10A, FIG. 10B). Macrophage Maea expression was determined to be required for BM macrophage development and erythropoiesis at steady state (FIG. 9D-FIG. 9F, FIG. 10C-FIG. 10H). Based on a previous study that depletion of macrophages could normalize polycythemia vera, treatment with anti-MAEA antibody will likely achieve similar effects.

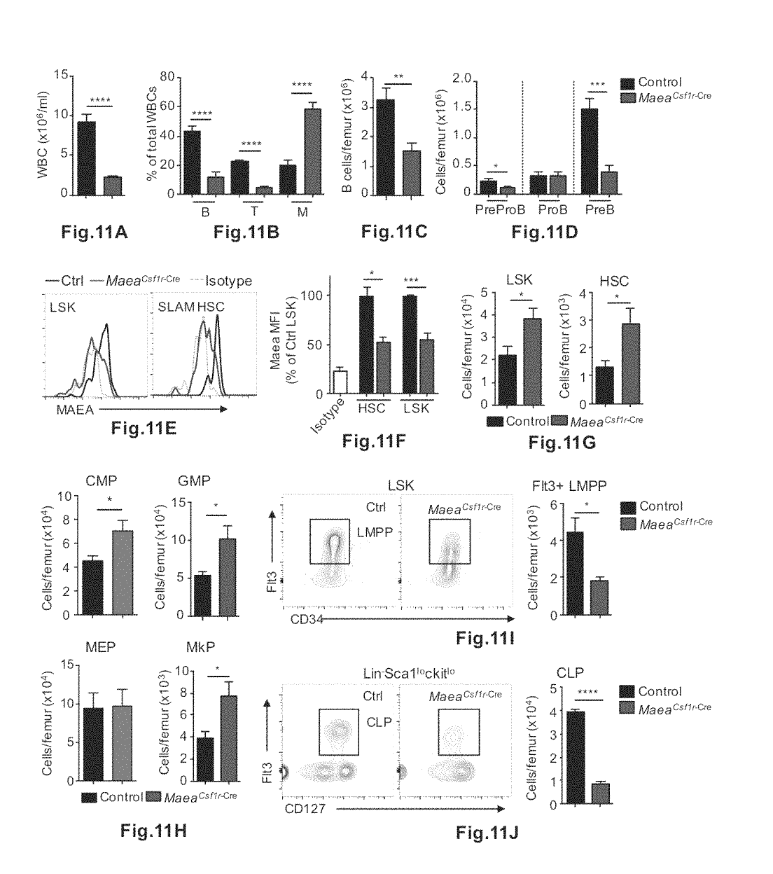

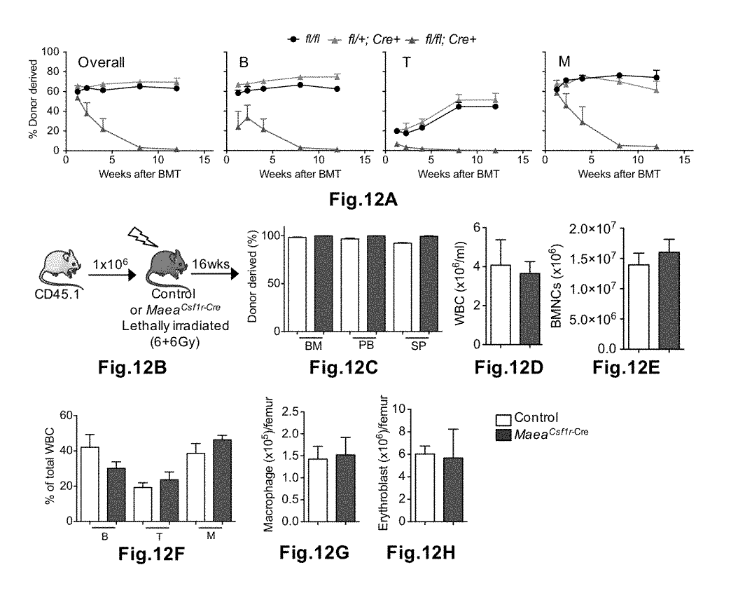

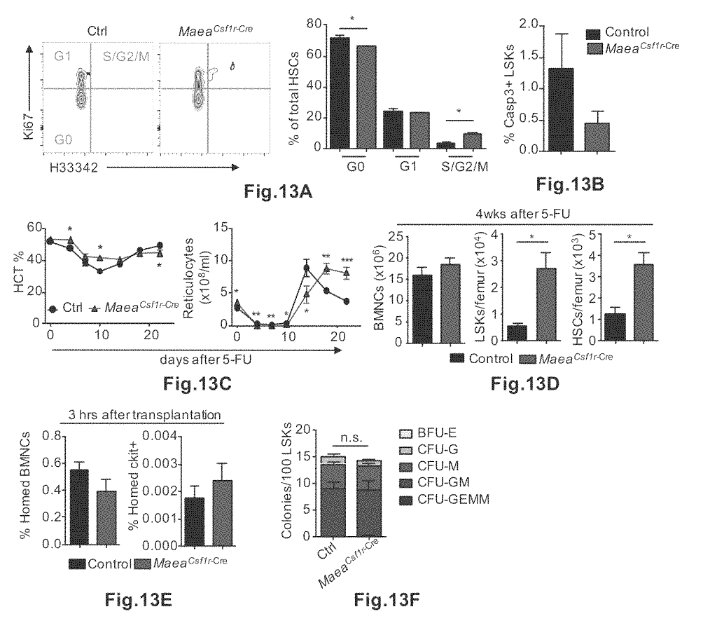

Unexpectedly, Maea^{Csflr-Cre} mice also exhibited marked reductions in circulating leukocytes (FIG. 11A), due to a loss of B and T lymphocytes (FIG. 11B-FIG. 11D). This is likely due to MAEA expression on bone marrow hematopoietic stem and progenitor cells (HSPCs) (FIG. 11E-FIG. 11F) and its involvement in lymphoid commitment from the HSPCs (FIG. 11G-FIG. 11J). Importantly, MEAE expression was also required for successful HSC engraftment after bone marrow transplantation (FIG. 12A), and this is not due to any microenvironmental defects (FIG. 12B-FIG. 12H). Without MAEA, HSCs are more actively cycling but do not show increased apoptosis (FIG. 13A-FIG. 13B). In addition, Maea-deficient HSCs regenerate (FIG. 13C-FIG. 13D), home to BM (FIG. 13E) and form colonies (FIG. 13F) comparably to control counterparts.

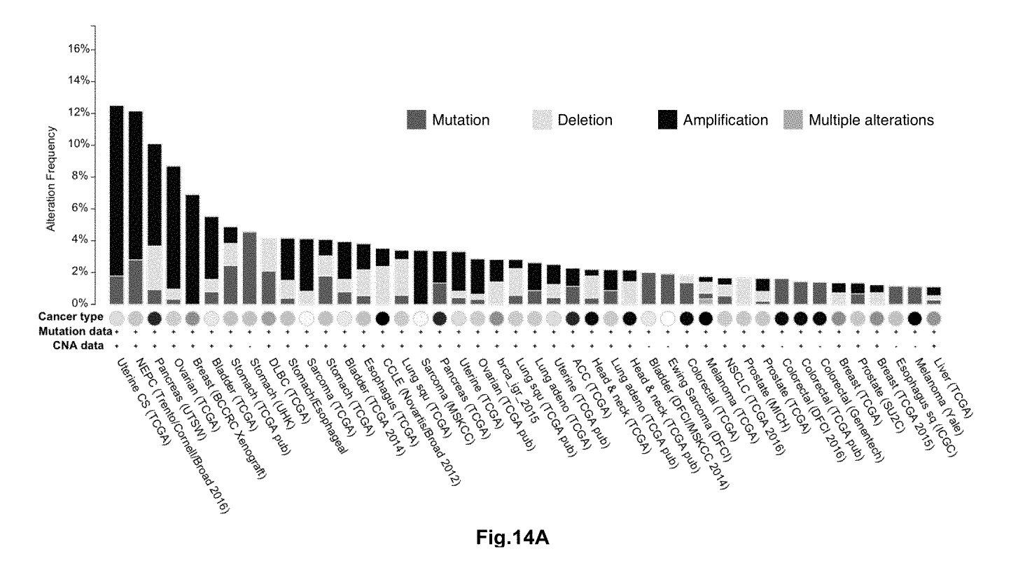

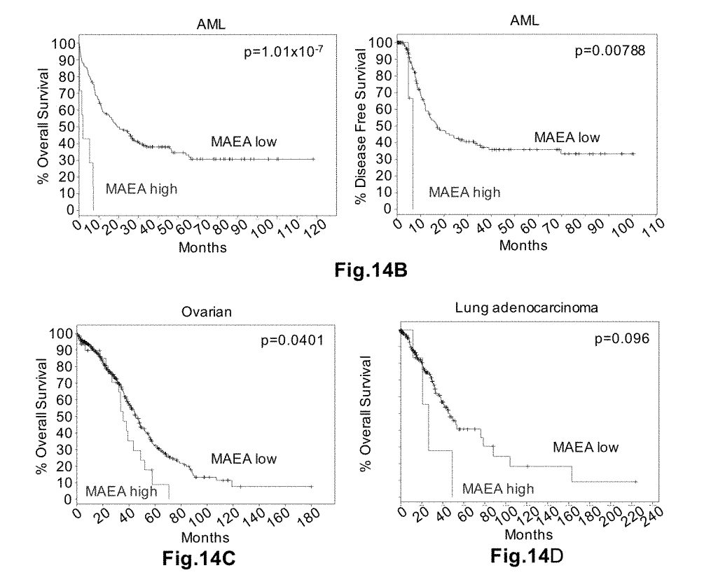

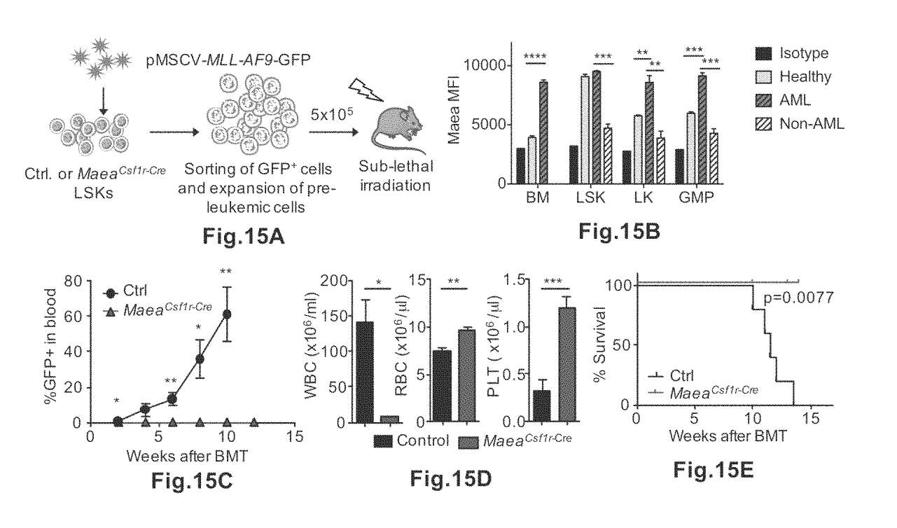

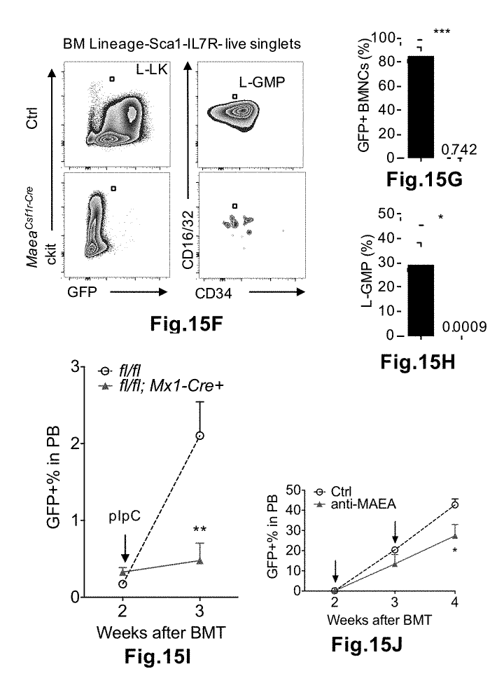

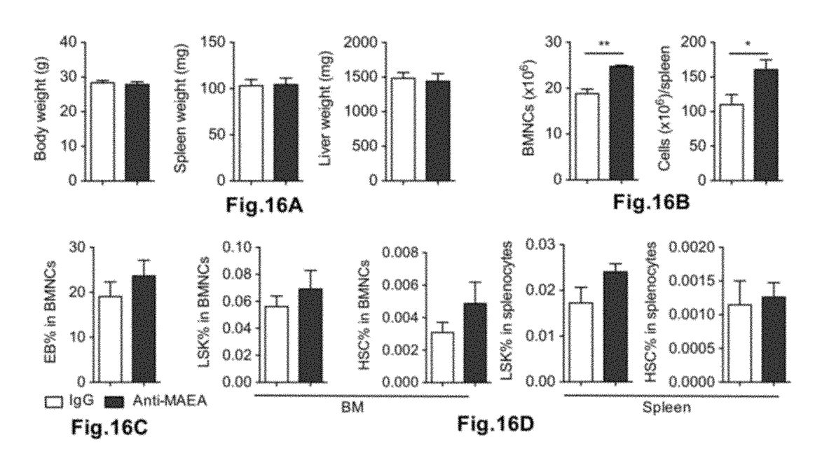

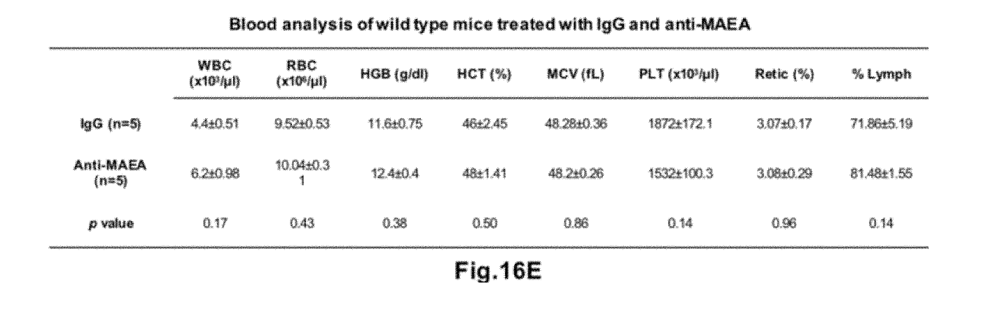

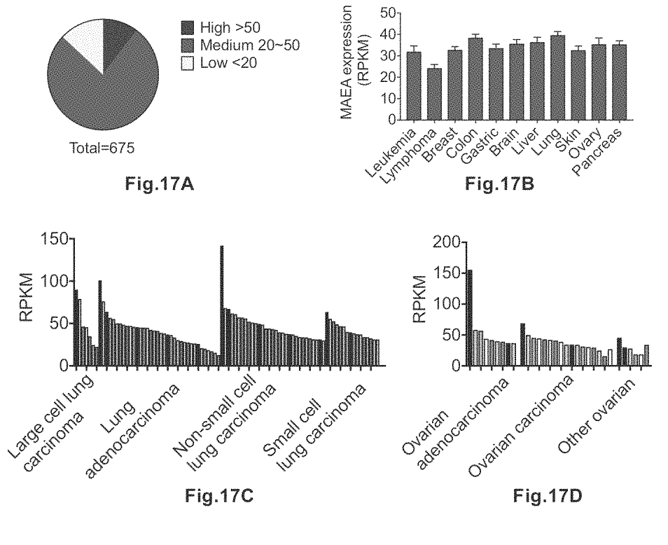

It was hypothesized that leukemia cells might hijack the same mechanism for their progression. Indeed, significant association of MAEA amplification mutations was found with many human cancer types (FIG. 14A), and MAEA up-regulation strongly correlated with poor prognosis in human AML patients (FIG. 14B-FIG. 14D). MAEA expression is also up-regulated in a murine model of acute myeloid leukemia (FIG. 15A-FIG. 15B). By genetically deleting MAEA expression from the AML cells using Csflr-Cre and Mx1-Cre, MAEA expression was shown to be required for AML engraftment and progression in vivo (FIG. 15C-FIG. 151). Importantly, treating AML-bearing mice with a polyclonal anti-MAEA antibody significantly reduced their circulating leukemia cells (FIG. 15J), but did not cause overt toxicity in healthy mice (FIG. 16). Lastly, analysis of human cancer cell lines revealed a broad expression of MAEA across cancer types (FIG. 17).

These studies indicate that MAEA is a novel adverse prognosis factor and drug target expressed on malignant hematopoietic and other cancer cells, and that MAEA is a target to promote cancer cell clearance by the host immune system.

### Example III

### Anti Maea Antibodies. Maea Expressed by Macrophages, but not Erythroblasts, Maintains Postnatal Bone Marrow Erythroblastic Islands

### Introduction

Red blood cell (RBC) homeostasis is tightly regulated by balanced production and clearance. Bone marrow (BM) erythroid precursors were first observed several decades ago in tight association with a central macrophage in a structure referred to as erythroblastic island (EI)¹. Macrophages regulate both normal and diseased erythropoiesis, including promotion of erythroid precursor survival and proliferation, iron homeostasis and transfer, and terminal maturation and enucleation²⁻⁵. These activities are promoted by direct interactions between the macrophages and erythroblasts^{6,7} via several proposed adhesion mechanisms including (macrophage: erythroblast) Vcam1: VLA-4^{8,9}, αV: Icam4¹⁰, or Maea: Maea⁷, CD163¹¹ and Palladin¹². However, the exact role of these adhesion molecules during in vivo adult erythropoiesis has not been determined.

Among these, Maea was originally identified as an adhesion molecule expressed by both macrophages and erythroblasts and suggested to mediate EI formation via its homophilic interactions^{7,13}. Targeted gene inactivation of Maea caused severe defects in fetal liver erythropoiesis and macrophage development¹⁴, but the perinatal lethality of Maea-null embryos has prevented detailed examination of its function in adult hematopoiesis. In this study, a conditional allele of Maea was generated. Maea was determined to play a critical role in adult BM macrophage development and EI function. Comparative analysis with Vcam1 deletion shows that Maea exerts a dominant role in the EI. Selective deletion of Maea in macrophage or erythroblast shows only disruption of BM erythropoiesis when Maea is deleted in macrophage, suggesting that Maea may not interact by homophilic interactions.

### Methods

Animals. Maea^{fl/fl} mice were generated as described below. Vcam1^{fl/fl} mice¹⁵ were kindly provided by Dr. Thalia Papayannopoulou and backcrossed to C57BL/6 strain for at least 10 generations. Csflr-Cre mice¹⁶ were a gift from Dr. Jeffrey W. Pollard (University of Edinburgh) and also backcrossed onto C57BL/6 background. CD169-Cre knockin mice were previously generated and described¹⁷. Epor-Cre mice¹⁸ were kindly provided by Dr. Ann Mullally (Dana-Farber/Brigham and Women's Hospital). C57BL/6 (CD45.2) and B16-Ly5.1 (CD45.1) mice were purchased from Charles River Laboratories (Frederick Cancer Research Center, Frederick, Md.)/NCI or the Jackson Laboratories (B6. SJL-Ptprc^{a} Pepc^{b}/BoyJ). R26-tdTomato (B6.Cg-Gt(ROSA)26Sor^{tm14(CAG-tdTomato)Hze}/J) and Mxl-Cre (B6.Cg-Tg(Mx1-cre)1Cgn/J) mice were from Jackson Laboratories. All animals were housed in specific pathogen-free barrier facility. All experimental procedures were approved by the Animal Care and Use Committee of Albert Einstein College of Medicine. All experiments were performed on mice of both genders with littermate controls from the same colony between 6-12 weeks of age.

Generation of Maea^{fl/fl} mice. The EuMMCR targeting vector PG00141_Z_1_G10 was purchased and electroporated into WW6 embryonic stem (ES) cells. After drug selection, resistant ES cell colonies were picked and screened by Southern blot analysis using 5′ and 3′ external probes. Correctly targeted ES cell clones were injected into C57BL/6 blastocysts and the resulting chimeric mice were bred with C57BL/6 animals to establish the Maea^{Targeted} line. Once the germline transmission was confirmed, the Maea^{Targeted} mice were crossed to Rosa26^{FLP1} mice (Jax stock#009086) to remove the LacZ/Neo cassette and generate the floxed allele Maea^{fl}. Both alleles were then backcrossed onto C57BL/6 background for at least 5 generations before crossing to the various Cre strains for functional studies. Genotyping was done by ear clip genomic DNA PCR using primers F1+R1+R2 and F2+R3+R4. Primer sequences are as follows: F1: gttcagcctcaggattcagg (SEQ ID NO:1); R1: atgagcaggggacctcaac (SEQ ID NO:2); R2: aactgatggcgagctcaga (SEQ ID NO:3); F2: caccagctcaggcagttaca (SEQ ID NO:4); R3: ccacaacgggttcttctgtt (SEQ ID NO:5); R4: cgggaagaagtgggattacc (SEQ ID NO:6).

Antibodies and flow cytometry. Purified goat anti-MAEA polyclonal antibody (I-20) was purchased from Santa Cruz and used at a 1:100 concentration. Conjugated donkey anti-goat IgG secondary antibodies were from Thermo Fisher and used at a 1:800 concentration. Fluorochrome-conjugated or biotinylated antibodies against mouse Gr-1 (Ly6C/G) (clone RB6-8C5), CD115 (clone AFS98), B220 (clone RA3-6B2), F4/80 (clone BM8), Vcam1 (clone 429), CD11b (clone M1/70), CD45 (clone 30-F11), Ter119 (clone TER-119), CD71 (clone R17217) and CD44 (clone IM7), CD45.1 (clone A20), CD45.2 (clone 104), were from BioLegend or eBiosciences. DAPI-negative singlets were analyzed for all live samples unless otherwise specified. Stained sample suspensions were acquired on a LSR II (BD) and results were analyzed and visualized by FlowJo (Tree Star). For sorting, samples were processed under sterile conditions and sorted on a BD FACSAria.

Generation of MAEA monoclonal antibody (mAb). BALB/c mice were immunized with a KLH-conjugated MAEA peptide that is part of the extracellular domain (AAQKN IDRET SHVTM VVAEL EKTLS GCPA (SEQ ID NO:7)) and boosted with the same peptide and recombinant MAEA protein (Novus#NBP2-23208). Hybridomas producing mAbs to human MAEA were generated by standard techniques from splenocytes fused to Ag8.653 or NSO^{bc12} myeloma cells¹⁹. Clone 92 (IgG2a) was firstly selected by ELISA screen as its mAbs recognized MAEA peptide/protein, but not human IgG. Subclone 92.25 was further selected and validated by FACS staining of wild type but not Maea^{CsflrCre} mouse BM cells due to only one amino acid difference between the human and mouse sequence in the antibody target region. mAbs were then concentrated and purified from concentrated hybridoma supernatant by Amicon Ultra-15 100K filters (Millipore) and NAb Protein A/G Spin kits (Thermo Scientific).

Complete blood count. Mice were bled ∥25 μl into an Eppendorf tube containing 2 μl of 0.5 M EDTA (Life Technologies) using heparinized micro-hematocrit capillary tubes (Fisherbrand) under isoflurane anesthesia. Blood was diluted 1:20 in PBS and analyzed on an Advia counter (Siemens).

In vivo clearance of RBCs. Long-term RBC clearance was assayed as previously described²⁰. Mice were given a single i.v. NHS sulfo-biotin (Thermo Scientific) injection (100 mg/kg), and the fraction of biotinylated RBCs was determined weekly from 1 μl of blood. Short-term clearance was assayed by i.v. injection of 2×10⁸ CFSE-labelled wild-type RBCs and monitored at indicated time points.

Splenectomy. Mice were splenectomized under 100 mg/kg ketamine and 10 mg/kg xylazine anesthesia as previously described²¹ and allowed to recover for 4 weeks before experiments.

Bone marrow transplantation. All recipient mice were lethally irradiated (600+600 cGy, at least 3 h apart) in a Shepherd Mark 1 irradiator. RBC-lysed bone marrow nucleated cells (1×10⁶, unless otherwise indicated) were then injected retro-orbitally under isoflurane anesthesia.

Colony-forming assays. Spleen BFU-E was assayed by plating 5×10⁵ RBC-lysed splenocytes in MethoCult M3436 (Stem Cell Technologies) according to the manufacturer's instructions and colonies were enumerated on day 10 of culture.

In vivo treatment. For hemolytic anemia induction, mice were injected i.p. with 40 mg/kg body weight PHZ (Sigma#114715) on day 0 and 1 of the experiment. For 5-FU challenge, a single dose (250 mg/kg body weight) of freshly made 5-FU was given i.v. to each mouse under isoflurane anesthesia. Mx/-Cre was induced by three doses of PolyI:C (Invivogen) injections every other day at 5 mg/kg i.p.. For antibody treatment, purified MAEA mAb and control IgG2a (BioXcell) were diluted in PBS and injected i.p. at 100 μg daily for 3 weeks.

Cell culture. In vitro terminal differentiation and enucleation of sorted polychromatic erythroblasts (EB-III) was done as previously described²². Briefly, EB-III were FACS sorted and cultured at <10⁶/ml in differentiation media composed of IMDM, 10% FBS, 1% BSA, 30ng/ml Epo (BioLegend), 0.2 mg/ml holo-transferrin (Sigma) and 10 μg/ml insulin (ThermoFisher) for 48 hours. At the end of the culture, cells were FACS analyzed by Ter119 and H33342 staining. In vitro phagocytosis assay using bone marrow (BMDMs) or spleen (SPDMs) derived macrophages was slightly modified from previously described²³. BMDMs or SPDMs were isolated by adherence from BM or splenic suspensions in macrophage media (RPMI1640 with 10%FBS, 10 mM HEPES and 10 ng/ml M-CSF) for 7 days^{24,25}. On day 7, BMDMs or SPDMs were harvested by gentle scraping and plated at 1×10⁵/well in 12-well plates for 24 hours in full macrophage media. The macrophages were then serum-starved for 2 hours before adding 10×10⁶ CFSE-labeled RBCs or 4×10⁴ CD45.1 BM cells as target. After 2˜3hours co-incubation, non-adherent cells were washed and macrophages were scraped for FACS analysis.

Quantification and statistical analysis. In each experiment, each mouse was analyzed as a biological replicate. Data visualization (shown as mean±s.e.m.) and statistical analysis were performed using Graphpad Prism 7. Unpaired Student's t-test was used to assess statistical significance when comparing two samples unless otherwise indicated.

### Results

Maea is required for adult BM macrophage development and EI niche formation. To examine Maea function in adult erythropoiesis, mice were generated with a floxed allele of Maea, which leads to a frame shift and non-sense mediated decay of Maea mRNA upon Cre-mediated recombination. Flow cytometry analysis using a MAEA-specific polyclonal antibody revealed that in adult BM mononucleated cells (BMNCs), Maea was highly expressed in the macrophages with minimal expression by monocytes, neutrophils or B cells (gated as previously described²⁶). Maea^{fl/fl} mice were intercrossed with a Csflr-Cre transgenic line¹⁶ to delete Maea in the monocytic-macrophage lineage. Csflr-Cre; Maea^{fl/fl} animals (henceforth Maea^{Csflr-Cre}) were born healthy and fertile, and survived into adulthood at expected Mendelian ratios, by contrast to the perinatal lethality reported in Maea-null mice¹⁴. Efficient MAEA-depletion on BM macrophages was confirmed by FACS analysis. The BM of Maea^{Csflr-Cre} mice exhibited a slight, but significant, reduction in cellularity. Further analysis revealed that their BM macrophage numbers represented 30% of wild-type levels. Maea^{Csflr-Cre} bones also appeared paler than controls, suggesting a reduced erythroid content in the marrow. Indeed, the number of BM erythroblasts was markedly reduced in Maea^{Csflr-Cre} BM compared to littermate controls. This is likely due to disruption of EI formation because there was a marked reduction in EIs (˜30% of control levels) formed in vivo in the Maea^{Csflr-Cre} BM. Profiling of the erythroblast maturation status revealed a partial block of differentiation at the polychromatic (EB-III) stage²⁷. These results support a critical role of Maea in adult BM erythroblastic island formation and functions.

Maea is dispensable for RBC enucleation. In contrast to the prior report on Maea-null mice¹⁴, peripheral blood anemia was not observed in young adult Maea^{Csflr-Cre} mice. Although a role for Maea in enucleation as also been suggested¹⁴, blood smears and FACS analyses revealed elevated CD71⁺ Ter119⁺ reticulocyte counts in Maea^{Csflr-Cre} animals but no nucleated RBCs in circulating blood. To investigate further this issue, polychromatic erythroblasts (EB-III) were sorted from BM of Maea^{Csflr-Cre} and control mice and enucleation rates were evaluated in vitro. Cultured Maea^{Csflr-Cre} derived erythroblasts enucleated at similar rate as those of controls. These results suggest that Maea expression is dispensable for postnatal RBC enucleation.

Maea regulates RBC dynamics during stress erythropoiesis. Control and Maea^{Csflr-Cre} mice were challenged with hemolytic anemia induced by the hemoglobin-oxidizing reagent phenylhydrazine (PHZ). There was a significant impairment of the reticulocytosis but not RBC or hematocrit recovery in Maea^{Csflr-Cre} mice. Similarly, reticulocytosis in Maea^{Csflr-Cre} mice after a single dose of cytotoxic agent 5-fluorouracil (5-FU) was significantly delayed while RBC and hematocrit showed an attenuated decline before a mild but significant delay in recovery. The attenuated early decline in hematocrit was consistent with previously described macrophage-depleted models²⁰, suggesting that Maea depletion caused macrophage defects were contributing to both the RBC production and clearance. Indeed, the RBC lifespan was significantly prolonged in Maea^{Csflr-Cre} animals. However, no phagocytosis defects in Maea^{Csflr-Cre} macrophages were detected, suggesting the RBC clearance defect likely reflected the overall reduction of macrophage numbers.

Differential roles of Maea in spleen and bone marrow macrophages. Interestingly, even though Csflr-Cre induced similarly efficient Maea deletion in splenic red pulp macrophages (RPMs), their numbers were not significantly altered. This suggests differential requirements of Maea in BM and spleen macrophage development or maintenance. Spleen EB numbers were not significantly altered, although profiling of their maturation revealed a similar partial block at the polychromatic stage. However, compensatory stress erythropoiesis was found in the spleen of Maea^{Csflr-Cre} mice as evidenced by splenomegaly and elevated burst-forming unit-erythroid (BFU-E) numbers.

To further dissect the requirement of Maea in BM and spleen erythropoiesis, splenectomy was performed on Maea^{Csflr-Cre} and littermate control mice. Splenectomized Maea^{Csflr-Cre} animals developed anemia over the course of 4 weeks while the control group maintained healthy peripheral blood counts, suggesting that in the context of Maea^{Csflr-Cre} animals, the extra-medullary erythropoiesis may mask the phenotype. The splenectomized control and Maea^{Csflr-Cre} mice were challenged with PHZ. A severely impaired recovery response was observed in Maea^{Csflr-Cre} mice. Blood smear again did not reveal any enucleation defect in the RBCs from splenectomized Maea^{Csflr-Cre} mice before or after PHZ treatment. These results further confirmed that Maea is critical for adult BM erythropoiesis but not RBC enucleation.

Dominant function of Maea over Vcam1 in EI niche formation. Vcam1 represents another adhesion molecule implicated in EI^{8,9,20}. To compare the role of Vcam1 with Maea, Vcam1 was deleted using Csflr-Cre. Interestingly, no defects were observed in BM macrophage or erythroblast numbers in steady state Vcam1^{Csflr-Cre} mice compared to the control animals, except for a minor trend towards reduced spleen erythroblasts. EB maturation as measured by CD44 expression and cell size also indicated a normal differentiation profile. The peripheral RBC compartment during steady state and after PHZ challenge was also normal, consistent with previous studies^{28,29}. These data indicate that Maea plays a dominant role in BM EI niche function while Vcam1 is dispensable for adult erythropoiesis in vivo.

Selective Maea deletion in macrophages, but not erythroblasts, impairs bone marrow erythropoiesis. Csflr-Cre broadly recombines in hematopoietic stem and progenitor cells (HSPCs), and thus may not discriminate between Maea function in macrophages or erythroblasts that descent from HSPCs. To delete selectively Maea in macrophages, Maea^{fl/fl} was crossed with CD169-Cre¹⁷, which does not target the erythroid lineage. Maea^{CD169-Cre} mice phenotypically mimicked the Maea^{Csflr-Cre} animals, with significant reduction of BM macrophage and erythroblast numbers, but no alterations in BM cellularity or circulating blood parameters. Further analyses revealed a similar defect in in vivo island formation and a partial block in BM EB maturation at the EB-III stage. Interestingly, while CD169-Cre also recombined efficiently in spleen RPMs, no significant change in RPM numbers was observed and EB numbers in the spleen of Maea^{CD169-Cre} mice were significantly increased suggesting ongoing extra-medullary erythropoiesis.

By contrast, when Maea^{fl/fl} was crossed with Epor-Cre, which recombines efficiently and selectively in erythroid progenitors¹⁸, Maea^{Epor-Cre} animals showed significant reductions in circulating RBC counts with increased mean corpuscular volume (MCV), but no significant change in BM macrophage and erythroblast numbers or in vivo erythroblast island formation. In addition, Maea^{Epor-cre} EB maturation showed a distinct profile with accumulation of the mature cells in the BM. No enucleation defect was observed in blood smear or in vitro EB-III culture. Analysis of the spleen did not reveal significant difference in macrophage or EB numbers of Maea^{Epor-cre} mice, despite efficient Epor-Cre recombination in spleen erythroid lineage. These results suggest that Maea acts in the macrophage, but not erythroblast, to mediate EI formation in adult BM, consistent with the previous report in an in vitro EI reconstitution setting¹⁴. The result also further indicates that Maea is dispensable for RBC enucleation but may play a cell autonomous function in RBC terminal maturation or egress in adult mice.

To investigate the role of macrophage or erythroblast conditional Maea deletion in stress erythropoiesis, the two models were challenged with PHZ-induced anemia. Surprisingly, Maea^{CD169-Cre} mice showed no significant difference in hematocrit recovery or reticulocytosis. This may be due to the fact that CD169-Cre recombines at a lower frequency (˜60%) in BM macrophages, and/or Csflr-Cre model is indeed a combinatory model of Maea-deletion in both the macrophages and erythroblasts. Indeed, BM of Maea^{CD169-Cre} mice showed constantly milder reduction of macrophage and EB numbers than Maea^{Csflr-Cre} mice at steady state and after PHZ. In contrast, Maea^{Epor-Cre} mice showed a faster decline in RBC content during the early days and a weaker reticulocyte output during the later recovery stages. The reduction of RBCs and reticulocytes but normal BM EB numbers after PHZ in Maea^{Epor-Cre} mice also further suggests that EB Maea expression may contribute cell autonomously to RBC terminal maturation or egress, but not in EI formation.

Postnatal Maea deletion or inhibition uncovers important functions in adult erythropoiesis. To evaluate the role of Maea in postnatal erythropoiesis and the maintenance of the EI niche, Maea was deleted using the Mx/-Cre line (Maea^{Mxl-Cre}) in which Cre-mediated recombination is inducible by Poly I:C injections. Radiation chimeras were generated by transplantation of the BM from Maea^{Mxl-cre} or littermate controls into wild-type mice to exclude potential complications from the BM microenvironment. Two months after transplantation, Cre recombination were induced by three Poly I:C injections. Three weeks after the first Poly I:C injection, BM macrophage and erythroblast numbers were significantly reduced in Maea^{Mxl-cre} animals, indicating that Maea is required cell-autonomously for the maintenance of BM macrophages and the EI niche during homeostasis.

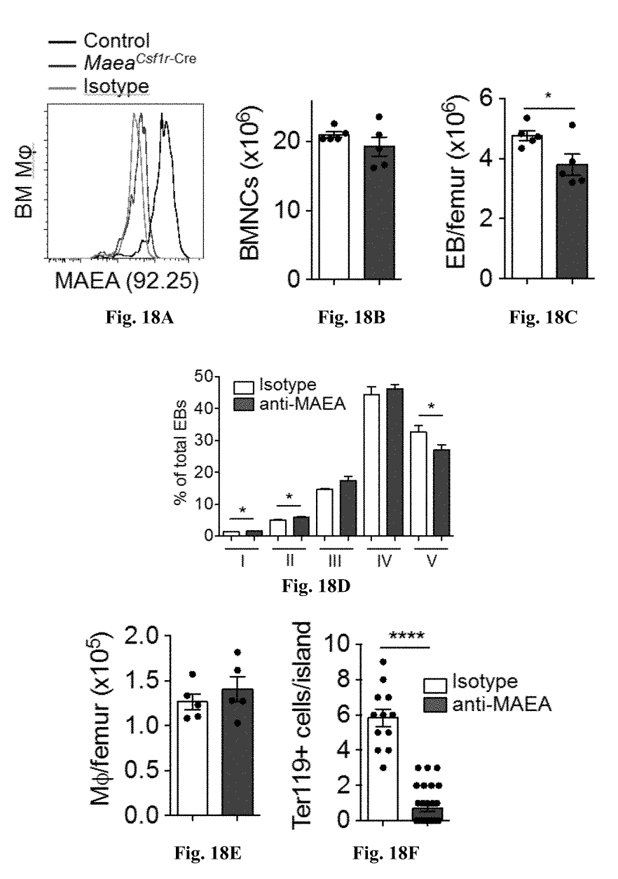

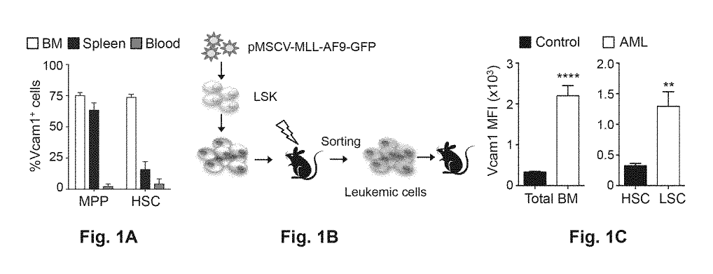

To investigate further the role of Maea in erythropoiesis, a novel monoclonal antibody was developed by immunization of Balb/c mice with a peptide corresponding to the extracellular domain of human MAEA. Clone 92.25 (IgG2a) was isolated, which interacts specifically with both murine and human MAEA, owing to the highly conserved MAEA amino acid sequence across species (FIG. 18A). Wild-type mice were treated with 92.25 or isotype control (100 μg daily 5 days a week for 3 weeks). Anti-MAEA significantly reduced erythroblast numbers in BM without affecting the total cellularity (FIG. 18B, 18C), but not in the spleen. The treatment also led to alterations in erythroblast differentiation similar to macrophage-selective Maea knockouts (FIG. 18D) without reductions of macrophage numbers (FIG. 18E). Furthermore, in vitro EI reconstitution assay showed that 92.25 significantly inhibited island formation (FIG. 18 F), clearly indicating that the EI phenotype from Maea deficiency does not originate solely from a defect of macrophage maturation and direct adhesion mediated via Maea is required for adult BM erythropoiesis.

### Discussion

The critical function of EI niche in erythropoiesis was initially suggested based on in vitro data^{6,7} and recently confirmed in vivo^{20,30}. However, in vivo studies using macrophage depletion cannot distinguish between the EI-dependent and EI-independent functions of the macrophage^{20,30}. Several adhesion mechanisms have been proposed to mediate EI formation, providing an ideal model for investigations of EI-specific functions^{7,8,10-12}. However, studies thus far have been largely based on in vitro EI formation assays or germline gene-deletions. Here it is shown that Maea is critical for adult BM EI formation and homeostatic erythropoiesis via multiple mechanisms. Both constitutive and induced Maea deletion result in severe reductions of BM macrophage numbers, indicating that Maea is required for BM macrophage homeostasis. Interestingly, spleen macrophages are not affected by Maea deletion, in line with recent reports indicating the independence and heterogeneity of tissue resident macrophages under steady state³¹⁻³³. Yet, RBC clearance is delayed in Maea^{Csflr-Cre} mice, suggesting a role of macrophages in the BM or other organs that might be affected by Maea-deletion in RBC clearance. Additionally, antibody inhibition disrupted EI formation in vivo and in vitro confirmed that Maea also directly mediates the adhesion of erythroblasts to macrophages to form EI^{13,14}. BM EB number and maturation profile is significantly impaired even when macrophage numbers are not affected (such as after anti-MAEA antibody inhibition), suggesting EI-specific functions of the macrophage in supporting EB differentiation. The macrophage and erythroid lineage-selective Maea deletion provides genetic evidence that Maea-mediated adhesion is unlikely the result of a homophilic interaction.

Maea appears dispensable for the enucleation of adult erythroblasts. The enucleation process of end-stage erythroid maturation is thought to be coordinated by the sorting and reassembly of nuclear, cytoplasmic and membrane contents among the resulting reticulocytes and pyrenocytes^{22,34,35}. Previous studies have suggested that Maea is associated with actin filaments, preferentially segregating into the extruding pyrenocytes and is required for EB enucleation^{14,35,36}. However, none of the present genetic deletion models have revealed any enucleation defect in vivo or in vitro, under steady state or after stress. One possibility is that the erythroid progenitors in previously reported Maea null embryos are so poorly differentiated due to defects upstream of the EI that they are not able to reach the enucleation stage^{14,36}. Alternatively, the residual expression of Maea in the present conditional knockout models may be masking the phenotype observed in the null embryos. It is also tempting to speculate that the enucleation process of fetal erythrocytes may be different from their adult counterpart, in parallel with their many other differences, such as the globin compositions^{37,38}.

The present studies provide genetic evidence that the contribution of Maea in BM EI is dominant compared to that of Vcam1, which when deleted using the same Csflr-Cre, is dispensable for macrophage development, in vivo EI function and erythroid recovery. Although Vcam1-mediated EI formation has commonly been observed in vitro^{8,39,40}, its requirement during in vivo erythropoiesis using genetic models has not been described^{28,29}. Studies using antibody inhibition in whole animals have suggested a contribution of Vcam1 in erythropoiesis²⁰, although Vcam1 expression and function outside of the EI—e.g. in endothelial cells⁴¹ or the hematopoietic stem and progenitor cell niche⁴²⁻⁴⁴—cannot be excluded. Since EI mediates erythropoiesis in health and disease²⁰′³°, the present study indicates that MAEA is a promising therapeutic target for erythropoietic disorders.

### REFERENCES

1 Bessis, M. [Erythroblastic island, functional unity of bone marrow]. Rev Hematol 13, 8-11 (1958).
2 Hom, J., Dulmovits, B. M., Mohandas, N. & Blanc, L. The erythroblastic island as an emerging paradigm in the anemia of inflammation. Immunol Res 63, 75-89, doi:10.1007/s12026-015-8697-2 (2015).
3 Jacobsen, R. N., Perkins, A. C. & Levesque, J. P. Macrophages and regulation of erythropoiesis. Curr Opin Hematol 22, 212-219, doi:10.1097/MOH.0000000000000131 (2015).
4 de Back, D. Z., Kostova, E. B., van Kraaij, M., van den Berg, T. K. & van Bruggen, R. Of macrophages and red blood cells; a complex love story. Front Physiol 5, 9, doi:10.3389/fphys.2014.00009 (2014).
5 Klei, T. R., Meinderts, S. M., van den Berg, T. K. & van Bruggen, R. From the Cradle to the Grave: The Role of Macrophages in Erythropoiesis and Erythrophagocytosis. Front Immunol 8, 73, doi:10.3389/fimmu.2017.00073 (2017).
6 Rhodes, M. M., Kopsombut, P., Bondurant, M. C., Price, J. O. & Koury, M. J. Adherence to macrophages in erythroblastic islands enhances erythroblast proliferation and increases erythrocyte production by a different mechanism than erythropoietin. Blood 111, 1700-1708, doi:10.1182/blood-2007-06-098178 (2008).
7 Hanspal, M. & Hanspal, J. S. The association of erythroblasts with macrophages promotes erythroid proliferation and maturation: a 30-kD heparin-binding protein is involved in this contact. Blood 84, 3494-3504 (1994).
8 Sadahira, Y., Yoshino, T. & Monobe, Y. Very late activation antigen 4-vascular cell adhesion molecule 1 interaction is involved in the formation of erythroblastic islands. J Exp Med 181, 411-415 (1995).
9 Hamamura, K. et al. A critical role of VLA-4 in erythropoiesis in vivo. Blood 87, 2513-2517 (1996).
10 Lee, G. et al. Targeted gene deletion demonstrates that the cell adhesion molecule ICAM-4 is critical for erythroblastic island formation. Blood 108, 2064-2071, doi:10.1182/blood-2006-03-006759 (2006).
11 Fabriek, B. O. et al. The macrophage CD163 surface glycoprotein is an erythroblast adhesion receptor. Blood 109, 5223-5229, doi:10.1182/blood-2006-08-036467 (2007).
12 Liu, X. S. et al. Disruption of palladin leads to defects in definitive erythropoiesis by interfering with erythroblastic island formation in mouse fetal liver. Blood 110, 870-876, doi:10.1182/blood-2007-01-068528 (2007).
13 Hanspal, M., Smockova, Y. & Uong, Q. Molecular identification and functional characterization of a novel protein that mediates the attachment of erythroblasts to macrophages. Blood 92, 2940-2950 (1998).
14 Soni, S. et al. Absence of erythroblast macrophage protein (Emp) leads to failure of erythroblast nuclear extrusion. J Biol Chem 281, 20181-20189, doi:10.1074/jbc.M603226200 (2006).
15 Koni, P. A. et al. Conditional vascular cell adhesion molecule 1 deletion in mice: impaired lymphocyte migration to bone marrow. J Exp Med 193, 741-754 (2001).
16 Qian, B. Z. et al. CCL2 recruits inflammatory monocytes to facilitate breast-tumour metastasis. Nature 475, 222-225, doi:10.1038/nature10138 (2011).
17 Karasawa, K. et al. Vascular-resident CD169-positive monocytes and macrophages control neutrophil accumulation in the kidney with ischemia-reperfusion injury. J Am Soc Nephrol 26, 896-906, doi:10.1681/ASN.2014020195 (2015).
18 Heinrich, A. C., Pelanda, R. & Klingmuller, U. A mouse model for visualization and conditional mutations in the erythroid lineage. Blood 104, 659-666, doi:10.1182/blood-2003-05-1442 (2004).
19 Sroubek, J. et al. The use of Bcl-2 over-expression to stabilize hybridomas specific to the HERG potassium channel. J Immunol Methods 375, 215-222, doi:10.1016/j.jim.2011.10.014 (2012).
20 Chow, A. et al. CD169(+) macrophages provide a niche promoting erythropoiesis under homeostasis and stress. Nat Med 19, 429-436, doi:10.1038/nm.3057 (2013).
21 Reeves, J. P., Reeves, P. A. & Chin, L. T. Survival surgery: removal of the spleen or thymus. Curr Protoc Immunol Chapter 1, Unit 1 10, doi:10.1002/0471142735.im0110s02 (2001).
22 Ji, P., Yeh, V., Ramirez, T., Murata-Hori, M. & Lodish, H. F. Histone deacetylase 2 is required for chromatin condensation and subsequent enucleation of cultured mouse fetal erythroblasts. Haematologica 95, 2013-2021, doi:10.3324/haemato1.2010.029827 (2010).
23 Majeti, R. et al. CD47 is an adverse prognostic factor and therapeutic antibody target on human acute myeloid leukemia stem cells. Cell 138, 286-299, doi:10.1016/j.ce11.2009.05.045 (2009).
24 Zhang, X., Goncalves, R. & Mosser, D. M. The isolation and characterization of murine macrophages. Curr Protoc Immunol Chapter 14, Unit 14 11, doi:10.1002/0471142735.im1401s83 (2008).
25 Davies, J. Q. & Gordon, S. Isolation and culture of murine macrophages. Methods Mol Biol 290, 91-103 (2005).
26 Chow, A. et al. Bone marrow CD169+macrophages promote the retention of hematopoietic stem and progenitor cells in the mesenchymal stem cell niche. J Exp Med 208, 261-271, doi:10.1084/jem.20101688 (2011).
27 Chen, K. et al. Resolving the distinct stages in erythroid differentiation based on dynamic changes in membrane protein expression during erythropoiesis. Proc Natl Acad Sci USA 106, 17413-17418, doi:10.1073/pnas.0909296106 (2009).
28 Ulyanova, T., Phelps, S. R. & Papayannopoulou, T. The macrophage contribution to stress erythropoiesis: when less is enough. Blood 128, 1756-1765, doi:10.1182/blood-2016-05-714527 (2016).
29 Ulyanova, T., Jiang, Y., Padilla, S., Nakamoto, B. & Papayannopoulou, T. Combinatorial and distinct roles of alpha(5) and alpha(4) integrins in stress erythropoiesis in mice. Blood 117, 975-985, doi:10.1182/blood-2010-05-283218 (2011).
30 Ramos, P. et al. Macrophages support pathological erythropoiesis in polycythemia vera and beta-thalassemia. Nat Med 19, 437-445, doi:10.1038/nm.3126 (2013).
31 Hashimoto, D. et al. Tissue-resident macrophages self-maintain locally throughout adult life with minimal contribution from circulating monocytes. Immunity 38, 792-804, doi:10.1016/j.immuni.2013.04.004 (2013).
32 Ginhoux, F. & Guilliams, M. Tissue-Resident Macrophage Ontogeny and Homeostasis. Immunity 44, 439-449, doi:10.1016/j.immuni.2016.02.024 (2016).
33 Hoeffel, G. & Ginhoux, F. Fetal monocytes and the origins of tissue-resident macrophages. Cell Immunol 330, 5-15, doi:10.1016/j.cellimm.2018.01.001 (2018).
34 Konstantinidis, D. G. et al. Signaling and cytoskeletal requirements in erythroblast enucleation. Blood 119, 6118-6127, doi:10.1182/blood-2011-09-379263 (2012).
35 C. et al. Mechanism of protein sorting during erythroblast enucleation: role of cytoskeletal connectivity. Blood 103, 1912-1919, doi:10.1182/blood-2003-03-0928 (2004).
36 Soni, S., Bala, S. & Hanspal, M. Requirement for erythroblast-macrophage protein (Emp) in definitive erythropoiesis. Blood Cells Mol Dis 41, 141-147, doi:10.1016/j.bcmd.2008.03.008 (2008).
37 Dame, C. & Juul, S. E. The switch from fetal to adult erythropoiesis. Clin Perinatol 27, 507-526 (2000).
38 Dzierzak, E. & Philipsen, S. Erythropoiesis: development and differentiation. Cold Spring Harb Perspect Med 3, a011601, doi:10.1101/cshperspect.a011601 (2013).
39 Yanai, N., Sekine, C., Yagita, H. & Obinata, M. Roles for integrin very late activation antigen-4 in stroma-dependent erythropoiesis. Blood 83, 2844-2850 (1994).
40 Toda, S., Segawa, K. & Nagata, S. MerTK-mediated engulfment of pyrenocytes by central macrophages in erythroblastic islands. Blood 123, 3963-3971, doi:10.1182/blood-2014-01-547976 (2014).
41 Yamashita, T. et al. The microenvironment for erythropoiesis is regulated by HIF-2alpha through VCAM-1 in endothelial cells. Blood 112, 1482-1492, doi:10.1182/blood-2007-11-122648 (2008).
42 Sturgeon, C. M. et al. Primitive erythropoiesis is regulated by miR-126 via nonhematopoietic Vcam-1+cells. Dev Cell 23, 45-57, doi:10.1016/j.devce1.2012.05.021 (2012).
43 Dutta, P. et al. Macrophages retain hematopoietic stem cells in the spleen via VCAM-1. J Exp Med 212, 497-512, doi:10.1084/jem.20141642 (2015).
44 Frenette, P. S., Subbarao, S., Mazo, I. B., von Andrian, U. H. & Wagner, D. D. Endothelial selectins and vascular cell adhesion molecule-1 promote hematopoietic progenitor homing to bone marrow. Proc Natl Acad Sci USA 95, 14423-14428 (1998).
45 Klijn C., et al. A comprehensive transcriptional portrait of human cancer cell lines. Nat Biotechnol. 2015 March; 33(3):306-12, Epub 2014 Dec. 8.

What is claimed is:

1. A method of treating a cancer in a subject comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) to treat a cancer in a subject, wherein the antibody or antibody fragment is specific for Vcam1 or Maea.

2. The method of claim 1, wherein the cancer is a cancer of one or more of bladder, breast, brain, colorectal, kidney, esophagus, gastrointestinal tract, liver, lung, ovarian, pancreas, prostate, skin, stomach, uterine, non-Hodgkin lymphoma, myelodysplatic syndrome, melanoma and a hematologic malignancy.

3. The method of claim 1, wherein the cancer is a hematologic malignancy.

4. The method of claim 3, wherein the hematologic malignancy is a myeloproliferative disease.

5. The method of claim 3, wherein the hematologic malignancy is acute myeloid leukemia (AML), chronic myeloid leukemia (CML), acute lymphoblastic leukemia (ALL), or chronic lymphocytic leukemia (CLL).

6. The method of claim 1, which comprises administering to the subject a combination of two or more of:
a) a blocking antibody to Vcam1 or an antibody fragment that blocks the activity of Vcam1, wherein the antibody or antibody fragment is specific for Vcam1;
b) a blocking antibody to Maea or an antibody fragment that blocks the activity of Maea, wherein the antibody or antibody fragment is specific for Maea;
c) one or more chemotherapeutic agents, and
d) one or more immune system enhancing agents;
wherein the combination includes at least a) or b).

7. The method of claim 6, wherein the one or more chemotherapeutic agents is selected from the group consisting of cytarabine, daunorubicin, idarubicin, mitoxantrone, cladribine, fludarabine, topotecan, etoposide, 6-thioguanine, hydroxyurea, prednisone, dexamethasone, methotrexate, 6-mercaptopurine, azacitidine, and decitabine.

8. The method of claim 6, wherein the one or more immune system enhancing agents is selected from the group consisting of an inhibitor of any of CD47, PD-1, PD-L1, CTLA-4, CD200, CD200R, LAG-3, TIM-3, VISTA, and TIGIT.

9. A method of inhibiting engraftment of leukemia cells in a subject, the method comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) to inhibit leukemia cell engraftment in a subject, wherein the antibody or antibody fragment is specific for Vcam1 or Maea.

10. The method of claim 9, wherein the leukemia cells are acute myeloid leukemia (AML), chronic myeloid leukemia (CML), acute lymphoblastic leukemia (ALL), or chronic lymphocytic leukemia (CLL) cells.

11. A method of enhancing the efficacy of cytarabine for treating a cancer in a subject, comprising administering to the subject an antibody or antibody fragment in an amount effective to inhibit the activity of Vascular cell adhesion molecule 1 (Vcam1) and/or an antibody or antibody fragment in an amount effective to inhibit the activity of Macrophage erythroblast attacher (Maea) in combination with cytarabine to enhance the efficacy of cytarabine for treating a cancer in a subject, wherein the antibody or antibody fragment is specific for Vcam1 or Maea.

12. The method of claim 11, wherein the cancer is one or more of acute myeloid leukemia (AML), chronic myeloid leukemia (CML), acute lymphoblastic leukemia (ALL), or chronic lymphocytic leukemia (CLL) and non-Hodgkin's lymphoma.

13. The method of claim 1, wherein the antibody or antibody fragment that inhibits the activity of Vcam1 is a blocking antibody to Vcam1 or an antibody fragment that blocks the activity of Vcam1.

14. The method of claim 1, wherein the antibody or antibody fragment that inhibits the activity of Maea is a blocking antibody to Maea or an antibody fragment that blocks the activity of Maea.

15. The method of claim 1, wherein the antibody is a monoclonal antibody.

16. The method of claim 1, wherein the antibody is a chimeric antibody, a humanized antibody or a human antibody.


SEQUENCE LISTING (7 sequences)

SEQ ID NO: 1 - DNA, 20 bp, Artificial Sequence
  forward primer 1 for Maea
gttcagcctcaggattcagg

SEQ ID NO: 2 - DNA, 19 bp, Artificial Sequence
  reverse primer 1 for Maea
atgagcaggggacctcaac

SEQ ID NO: 3 - DNA, 19 bp, Artificial Sequence
  reverse primer 2 for Maea
aactgatggcgagctcaga

SEQ ID NO: 4 - DNA, 20 bp, Artificial Sequence
  forward primer 2 for Maea
caccagctcaggcagttaca

SEQ ID NO: 5 - DNA, 20 bp, Artificial Sequence
  reverse primer 3 for Maea
ccacaacgggttcttctgtt

SEQ ID NO: 6 - DNA, 20 bp, Artificial Sequence
  reverse primer 4 for Maea
cgggaagaagtgggattacc

SEQ ID NO: 7 - protein, 29 aa, Homo sapiens
AAQKNIDRETSHVTMVVAELEKTLSGCPA